(19) United States
(12) Patent Application Publication
(10) Pub. No.: US 2018/0160910 A1
(43) Pub. Date: Jun. 14, 2018

(54) MEDICAL SUPPORT DEVICE, METHOD THEREOF, AND MEDICAL SUPPORT SYSTEM

(71) Applicant: SONY CORPORATION, Tokyo (JP)

(72) Inventors: Yasuaki TAKAHASHI, Kanagawa (JP); Hiroshi ICHIKI, Kanagawa (JP); Daisuke KIKUCHI, Kanagawa (JP); Yukihiro NAKAMURA, Kanagawa (JP); Tsuneo HAYASHI, Tokyo (JP); Takami MIZUKURA, Kanagawa (JP); Takeshi MIYAI, Kanagawa (JP); Daisuke TSURU, Chiba (JP)

(73) Assignee: SONY CORPORATION, Tokyo (JP)

(21) Appl. No.: 15/577,826

(22) PCT Filed: Jun. 3, 2016
(86) PCT No.: PCT/JP2016/066544

§ 371 Date: Nov. 29, 2017

(30) Foreign Application Priority Data
Jun. 19, 2015 (JP) 2015-124106

(51) Int. Cl. A61B 5/01; A61B 90/00; A61B 5/02; A61B 18/08; A61B 1/313; A61B 1/06; A61B 1/04

(52) U.S. Cl. CPC A61B 5/015; A61B 90/37; A61B 5/02042; A61B 2018/00041; A61B 1/313; A61B 1/0669; A61B 1/04; A61B 18/08

(57) ABSTRACT

The present technology relates to a medical support device, a method thereof, and a medical support system, which make it possible to notify of more various events.

In the present technology, an event in a living body is detected on the basis of change in time of temperature of a surgical site or around the surgical site, and notification information notifying according to the detected event in the living body is generated. The present technology may be applied to, for example, the medical support device, an endoscope device, a microscope device, a computer controlling the devices, or a medical support system including a plurality of devices and the like.

### TECHNICAL FIELD

The present technology relates to a medical support device, a method thereof, and a medical support system, and relates especially to a medical support device, a method thereof, and a medical support system capable of notifying of more various events.

### BACKGROUND ART

Conventionally, it is considered to measure temperature distribution in a living body subjected to medical care at the time of a medical practice such as surgery, and generate a temperature distribution image to use (refer to, for example, Patent Document 1).

### CITATION LIST

### Patent Document

### Patent Document 1: Japanese Patent Application Laid-Open No. 2001-286436

### SUMMARY OF THE INVENTION

### Problems to be Solved by the Invention

However, according to the method disclosed in Patent Document 1, merely the temperature distribution image is obtained. Therefore, a user such as a surgeon must obtain information by analyzing the displayed temperature distribution image, which requires complicated work and might reduce medical work efficiency. In addition, knowledge and techniques for correctly obtaining information from the temperature distribution image might be required.

Furthermore, since various events might occur at the time of medical care such as surgery, it might be difficult for the surgeon and the like to sufficiently easily and correctly grasp the events only by displaying the temperature distribution image as disclosed in Patent Document 1. That is, there is a possibility that the medical care cannot be fully supported.

The present technology is proposed in view of such a situation, and an object thereof is to make it possible to notify of more various events.

### Solutions to Problems

A medical support device according to the present technology is a medical support device provided with a detection unit which detects an event in a living body on the basis of change in time of temperature of a surgical site or around the surgical site and a notification information generation unit which generates notification information for notifying in accordance with the event in the living body detected by the detection unit.

The detection unit may detect the event in the living body on the basis of change in time of a temperature distribution image of the surgical site or around the surgical site.

The detection unit may detect a state to be warned as the event in the living body on the basis of the change in time of the temperature distribution image, and the notification information generation unit may generate a warning image to warn about the state.

The notification information generation unit may generate the warning image superimposed on a captured image of the surgical site or around the surgical site to be displayed.

The detection unit may detect occurrence of bleeding on the basis of the change in time of the temperature distribution image, and the notification information generation unit may generate, as the warning image, one or more of an image notifying of the occurrence of the bleeding, an image notifying of the number of times of occurrence of the bleeding, an image illustrating an occurrence site in a case where the occurrence site of the bleeding is located within a range of the captured image, and an image illustrating a direction of the occurrence site in a case where the occurrence site of the bleeding is located outside the range of the captured image.

The detection unit may detect that a surgical tool after use is in a state of predetermined temperature or higher on the basis of the change in time of the temperature distribution image, and the notification information generation unit may generate, as the warning image, one or more of an image notifying of presence of the surgical tool in the state of the predetermined temperature or higher, an image notifying of the number of surgical tools, an image illustrating the surgical tool in a case where the surgical tool is located within a range of the captured image, and an image illustrating a direction of the surgical tool in a case where the surgical tool is located outside the range of the captured image.

The detection unit may detect a result of hemostasis on the basis of the change in time of the temperature distribution image, and the notification information generation unit may generate, as the warning image, one or more of an image notifying of the result of the hemostasis, an image notifying of the number of sites where the hemostasis is performed, an image illustrating a site where the hemostasis is performed in a case where the hemostasis is performed within a range of the captured image, and an image illustrating a direction in which the hemostasis is performed in a case where the hemostasis is performed outside the range of the captured image.

The detection unit may detect a damaged site of the surgical site or around the surgical site due to contact of the surgical tool not in use on the basis of the change in time of the temperature distribution image, and the notification information generation unit may generate, as the warning image, one or more of an image notifying of occurrence of the damaged site, an image notifying of the number of damaged sites, an image illustrating the damaged site in a case where the damaged site is located within a range of the captured image, and an image illustrating a direction of the damaged site in a case where the damaged site is located outside the range of the captured image.

The detection unit may detect a site in a state of predetermined temperature or higher of the surgical site or around the surgical site due to use of the surgical tool on the basis of the change in time of the temperature distribution image, and the notification information generation unit may generate, as the warning image, one or more of an image notifying of occurrence of the site in the state of the predetermined temperature or higher, an image notifying of the number of sites in the state of the predetermined temperature or higher, an image illustrating an occurrence position of the site in the state of the predetermined temperature or higher in a case where the site in the state of the predetermined temperature or higher is located within a range of the captured image, and an image illustrating an occurrence direction of the site in the state of the predetermined temperature or higher in a case where the site in the state of the predetermined temperature or higher is located outside the range of the captured image.

The detection unit may detect change in state of the living body as the event in the living body on the basis of the change in time of the temperature distribution image, and the notification information generation unit may generate a diagnosis image notifying of a diagnosis result based on the change in state.

A heating unit which heats the living body is further provided, and the detection unit may detect the change in state of the living body after being heated by the heating unit.

The detection unit may perform detection of a subsegment of a liver, detection of a lesion, detection of a blood vessel, detection of a lymphatic vessel, identification of bone and nerve, or detection of a cancer cell, and the notification information generation unit may generate an image illustrating the subsegment, the lesion, the blood vessel, the lymphatic vessel, the bone, the nerve, or the cancer cell detected by the detection unit so as to be identifiable superimposed on a captured image of the surgical site to be displayed as the diagnosis image.

The detection unit may further detect the event in the living body on the basis of a sensing result other than the change in time of the temperature of the surgical site or around the surgical site.

The detection unit may detect the event in the living body on the basis of the change in time of the temperature of the surgical site or around the surgical site and a stereo image of the surgical site or around the surgical site.

A temperature sensor which detects temperature of the surgical site or around the surgical site is further provided, and the detection unit may detect the event in the living body on the basis of the change in time of the temperature of the surgical site or around the surgical site detected by the temperature sensor.

A display unit which displays an image of the notification information generated by the notification information generation unit may be further provided.

A control unit which controls the surgical tool according to the event in the living body detected by the detection unit may be further provided.

The control unit may control temperature of gas supplied into the living body or brightness of illumination light emitted into the living body.

A medical support method according to the present technology is a medical support method of a medical support device including detecting an event in a living body on the basis of change in time of temperature of a surgical site or around the surgical site, and generating notification information notifying according to the detected event in the living body.

A medical support system according to the present technology is a medical support system including a temperature sensor, a medical support device, and a display device, the temperature sensor configured to measure temperature of a surgical site or around the surgical site, the medical support device including a detection unit which detects an event in a living body on the basis of change in time of temperature of the surgical site or around the surgical site measured by the temperature sensor, and a notification information generation unit which generates notification information notifying according to the event in the living body detected by the detection unit, and the display device configured to display an image of the notification information generated by the notification information generation unit.

In the medical support device and method according to the present technology, an event in a living body is detected on the basis of change in time of temperature of a surgical site or around the surgical site, and notification information notifying according to the detected event in the living body is generated.

In a medical support system according to the present technology, the temperature of the surgical site or around the surgical site is measured by a temperature sensor, and a detection unit of the medical support device detects the event in the living body on the basis of the change in time of the temperature of the surgical site or around the surgical site measured by the temperature sensor, notification information notifying according to the detected event in the living body is generated, and an image of the notification information is displayed by a display device.

### Effects of the Invention

According to the present technology, medical care may be supported. Also, according to the present technology, it is possible to notify of more various events.

### BRIEF DESCRIPTION OF DRAWINGS

FIG. 1 is a view illustrating a main configuration example of an endoscopic surgery support system.

FIG. 2 is a view for illustrating a range of sensing.

FIG. 3 is a block diagram illustrating a main configuration example of a monitoring device and the like.

FIG. 4 is a flowchart for illustrating an example of a flow of a warning process.

FIG. 5 is a view for illustrating an example of change in time of temperature distribution.

FIG. 6 is a flowchart for illustrating an example of a flow of a bleeding detection process.

FIG. 7 is a view for illustrating an example of a display image.

FIG. 8 is a view for illustrating an example of change in time of temperature distribution.

FIG. 9 is a flowchart for illustrating an example of a flow of an after-use caution-needed state detection process.

FIG. 10 is a view for illustrating an example of a display image.

FIG. 11 is a view for illustrating an example of a display image.

FIG. 12 is a block diagram illustrating a main configuration example of a monitoring device and the like.

FIG. 13 is a flowchart for illustrating an example of a flow of a warning process.

FIG. 14 is a flowchart for illustrating an example of a flow of an after-use caution-needed state detection process.

FIG. 15 is a block diagram illustrating a main configuration example of a monitoring device and the like.

FIG. 16 is a view for illustrating a main configuration example of a liver.

FIG. 17 is a view for illustrating an example of a subsegment detection method.

FIG. 18 is a flowchart for illustrating an example of a flow of a diagnosis process.

FIG. 19 is a view for illustrating an example of a display image.

FIG. 20 is a block diagram illustrating a main configuration example of a monitoring device and the like.

FIG. 21 is a flowchart for illustrating an example of a flow of a warning control process.

FIG. 22 is a view for illustrating an example of a display image.

FIG. 23 is a flowchart for illustrating an example of a flow of a temperature abnormality detection process.

FIG. 24 is a flowchart for illustrating an example of a flow of a control process.

FIG. 25 is a block diagram illustrating a main configuration example of a computer.

### MODE FOR CARRYING OUT THE INVENTION

Modes for carrying out the present disclosure (hereinafter, referred to as embodiments) are hereinafter described. Meanwhile, the description is given in the following order.

1. First Embodiment (Endoscopic Surgery Support System)

2. Second Embodiment (Endoscopic Surgery Support System)

3. Third Embodiment (Endoscopic Surgery Support System)

4. Fourth Embodiment (Endoscopic Surgery Support System)

5. Fifth Embodiment (Endoscopic Surgery Support System)

6. Sixth Embodiment (Computer)

### 1. First Embodiment

<Monitoring of Abnormalities and the Like Occurring in Surgical Site and around Surgical Site>

Conventionally, in endoscopic surgery to perform surgery using an endoscope (for example, laparoscopic surgery and the like), and microscopic surgery to perform surgery using a microscope, a surgeon who performs surgery carries out operation while checking a captured image of a surgical field (field of view including the surgical site where the operation is carried out) using a monitor.

Because the surgeon and the like concentrate on the operation, it is difficult to visually grasp all of abnormalities occurring in the surgical site and around the surgical site, change in state there and the like.

It is considered to give more information by providing an image other than the captured image such as a temperature distribution image disclosed in Patent Document 1 to the surgeon and the like, but in order to obtain the information from the temperature distribution image, tangled operation such as analysis might be necessary, and in addition to that, knowledge and technique for analyzing the temperature distribution image might be necessary.

Also, although various events might occur in the surgery and the like, the information obtained only from the temperature distribution is limited, and it might be difficult for the surgeon and the like to grasp the events sufficiently easily and accurately. That is, it might be impossible to sufficiently support medical care only by presenting the temperature distribution image.

Therefore, an object is to enable notification of more various events in the medical care such as the surgery.

<Endoscopic Surgery Support System>

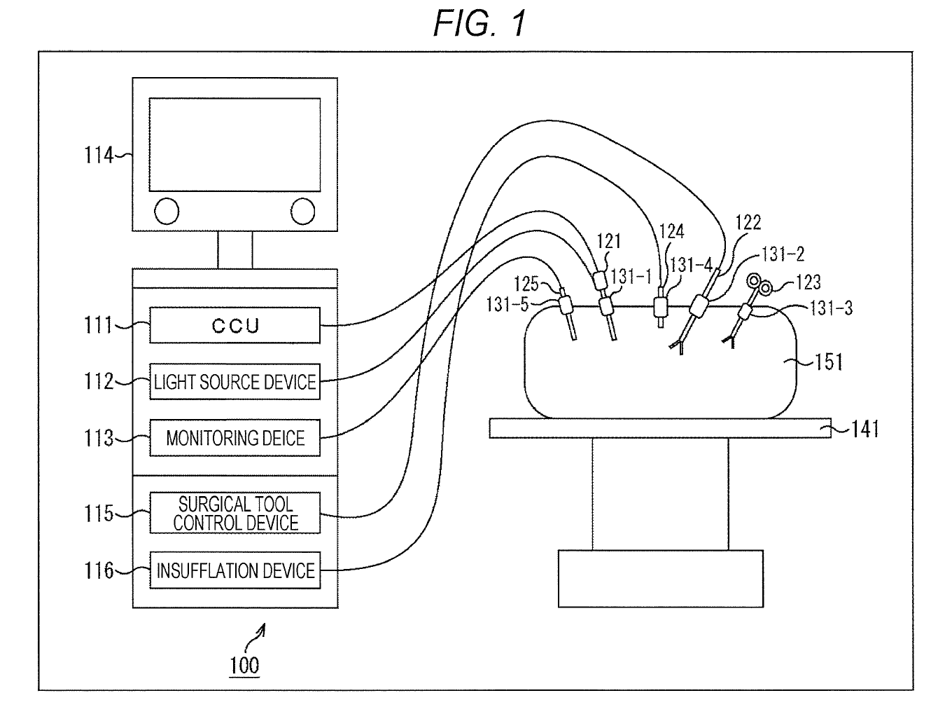

FIG. 1 is a view illustrating a main configuration example of an endoscopic surgery support system being an embodiment of a medical support system to which the present technology is applied.

In FIG. 1, an endoscopic surgery support system 100 is a system which supports surgery used by a surgeon and the like in endoscopic surgery (for example, laparoscopic surgery and the like). The endoscopic surgery is the surgery to open several small holes on a human body (for example, an abdomen and the like) of a patient subjected to the surgery, insert an endoscope and the like therefrom, and perform operation while looking at a monitor. The endoscopic surgery has a characteristic that this is less invasive than abdominal surgery and has less stress to the patient, so that early recovery is expected.

On a body surface of a patient 151 on a patient bed 141, a small hole is provided in the vicinity of a surgical site, and a trocar as a hole opening tool is attached to the hole so that the hole is kept open (an opening is formed). In a case of an example in FIG. 1, five holes are provided on a living body of the patient 151, and trocars 131-1 to 131-5 are attached to them to form the openings. In the following description, in a case where it is not required to distinguish the trocars 131-1 to 131-5 from one another to describe, they are referred to as the trocars 131.

In the endoscopic surgery, a surgical tool used in the surgery (for example, an electric scalpel, scissors, tweezers, forceps and the like) is inserted into the living body of the patient 151 from the opening by the trocar 131. In the example in FIG. 1, an endoscope 121, an energy device 122, forceps 123, an insufflation needle 124, and a monitoring sensor 125 are used as the surgical tools.

As described above, the endoscope 121 is the device which images the surgical site and the like. The endoscope 121 including an image sensor (an imaging element) irradiates an inside of the living body of the patient 151 (the surgical site and around the surgical site) with light supplied from a light source device 112 and utilizes the light to image. The endoscope 121 supplies information regarding a captured image obtained by imaging (for example, captured image data and the like) to a CCU 111. In the example in FIG. 1, the endoscope 121 is inserted into the living body of the patient 151 from the opening formed by the trocar 131-1.

The energy device 122 is the surgical tool which utilizes electric energy such as the electric scalpel, for example. For example, in a case of the electric scalpel, the energy device 122 may apply high-frequency current to the surgical site and generate Joule heat by a load or contact resistance at that time. This heat instantaneously heats up a cell to explode and evaporate, thereby causing an incision effect. Also, this heat evaporates moisture in the cell and coagulates protein, thereby causing a coagulation effect. In the example in FIG. 1, the energy device 122 is inserted into the living body of the patient 151 from the opening formed by the trocar 131-2.

The forceps 123 are tools for gripping the living body (for example, tissue) and the like. The forceps 123 having an appearance like scissors with a stopper attached at hand, for example, may maintain a state of pinching the living body and the like. The forceps 123 are used for various purposes such as pinching, tugging, crushing, opening, scooping, blocking and the like. In the example in FIG. 1, the forceps 123 are inserted into the living body of the patient 151 from the opening formed by the trocar 131-3.

The insufflation needle 124 supplies gas (carbon dioxide gas and the like) into the living body of the patient 151 (a space around the surgical site and the like) and sucks gas in the living body of the patient 151. The insufflation needle 124 is connected to an insufflation device 116 via a predetermined tube, and the gas supplied into the living body of the patient 151 is supplied from, for example, the insufflation device 116. Also, the gas sucked from the living body of the patient 151 is supplied to, for example, the insufflation device 116. In the example in FIG. 1, the insufflation needle 124 is inserted into the body of the patient 151 from the opening formed by the trocar 131-4.

The monitoring sensor 125 performs sensing (measurement) on parameters to be monitored regarding a target area. The area (target area) where the sensing is performed is also referred to as a sensing area. The monitoring sensor 125 makes at least the surgical site or around the surgical site the sensing area and measures temperature thereof for an arbitrary period. More specifically, the monitoring sensor 125 measures planar or spatial temperature distribution of at least the surgical site or around the surgical site for an arbitrary period. That is, it may also be said that the monitoring sensor 125 measures change in time of the temperature (more specifically, the planar or spatial temperature distribution) of the surgical site or around the surgical site where the medical care is performed.

The monitoring sensor 125 supplies information regarding a measurement result (sensing result) (for example, data obtained by the sensing and the like) to the monitoring device 113. That is, the monitoring sensor 125 supplies at least the planar or spatial temperature distribution during the period in which the measurement is performed (that is, the change in time of the planar or spatial temperature distribution during the period) to the monitoring device 113. In the example in FIG. 1, the monitoring sensor 125 is inserted into the living body of the patient 151 from the opening formed by the trocar 131-5.

As illustrated in FIG. 1, the endoscopic surgery support system 100 includes, for example, the camera control unit (CCU) 111, the light source device 112, the monitoring device 113, an output device 114, a surgical tool control device 115, the insufflation device 116 and the like.

The CCU 111 controls driving of the endoscope 121. For example, the CCU 111 supplies information regarding control to the endoscope 121 and controls driving of an observation optical system (for example, a lens, a diaphragm, an imaging element and the like) of the endoscope 121. The CCU 111 may also obtain the information regarding the captured image imaged through the observation optical system of the endoscope 121. For example, the CCU 111 may obtain the information regarding the captured image of the inside of the living body (the surgical site and around the surgical site) of the patient 151 imaged by the endoscope 121. The CCU 111 may also perform signal processing such as image processing on the captured image data included in the obtained information.

The light source device 112 is connected to the endoscope 121 via a light guide cable and the like and supplies a light source required for imaging by the endoscope 121 to the endoscope 121 via the light guide cable and the like.

The monitoring device 113 is an embodiment of a medical support device to which the present technology is applied. The monitoring device 113 detects an event in the living body on the basis of the information regarding the sensing result obtained via the monitoring sensor 125. The monitoring device 113 also generates notification information for notifying according to the detected event in the living body. For example, on the basis of the temperature distribution in the sensing area during the period in which the monitoring sensor 125 performs the measurement (that is, the change in time of the temperature distribution of the sensing area in that period), the monitoring device 113 detects the event in the living body to be warned. The monitoring device 113 also generates warning information for warning about the event in the living body as the notification information.

The output device 114 including, for example, a monitor for displaying an image, a speaker for outputting sound and the like outputs the information supplied from other devices such as the monitoring device 113 as an image or sound. For example, the output device 114 outputs (displays the image and outputs the sound) the captured image imaged by the endoscope 121 and the notification information (image and sound) generated by the monitoring device 113.

The surgical tool control device 115 controls driving of the energy device 122. For example, the surgical tool control device 115 supplies high-frequency current and the like to the energy device 122 so that the energy device 122 may resect and the like an affected site by electric heat thereof.

The insufflation device 116 supplies gas (for example, carbon dioxide gas and the like) to the insufflation needle 124 via a predetermined tube and supplies the same from the insufflation needle 124 to the inside of the living body (the surgical site or around the surgical site) of the patient 151 (supplies gas) to inflate the living body (around the surgical site). As a result, it is possible to secure a workable space in the living body (around the surgical site). The insufflation device 116 may also draw (suck) the gas (for example, carbon dioxide gas and the like) in the living body of the patient 151 from the insufflation needle 124 via the predetermined tube.

Meanwhile, a configuration of the endoscopic surgery support system 100 illustrated in FIG. 1 is an example, and the configuration of the endoscopic surgery support system 100 is not limited to the example in FIG. 1. For example, any type of the surgical tool may be used in the endoscopic surgery. For example, the endoscopic surgery support system 100 may be provided with the surgical tool other than the endoscope 121, the energy device 122, the forceps 123, the insufflation needle 124, and the monitoring sensor 125 described above. It is also possible that a part of the above-described surgical tools is not used.

Also, in FIG. 1, one endoscope 121, one energy device 122, one pair of forceps 123, one insufflation needle 124, and one monitoring sensor 125 are illustrated, but the numbers thereof are arbitrary. For example, the endoscopic surgery support system 100 may be provided with a plurality of at least one of them. It is also possible that the numbers of various types of surgical tools such as the endoscope 121, the energy device 122, the forceps 123, the insufflation needle 124, and the monitoring sensor 125 are not the same.

The endoscope 121 is connected to the CCU 111 so as to be able to communicate with each other. This communication may be wired communication, wireless communication, or both of them.

Also, the endoscope 121 may include a plurality of image sensors (imaging elements). In this case, a part of the image sensors may perform sensing of light of a type (for example, frequency band) different from that of at least another part of the image sensors. For example, the endoscope 121 may include the image sensor which performs the sensing of visible light and the image sensor which performs the sensing of infrared light. Also, for example, the endoscope 121 may include the image sensor which obtains a colored (for example, RGB) captured image and the image sensor which obtains a monochrome captured image. Also, among the plurality of image sensors included in the endoscope 121, characteristics such as the number of pixels and a pixel size of a part of the image sensors may be different from those of at least another part of the image sensors. The endoscope 121 may also include the image sensor capable of performing the sensing of light of a plurality of types (frequency bands). For example, the endoscope 121 may include one or more image sensors capable of performing the sensing of a plurality of types of light. Also, the endoscope 121 may include a plurality of image sensors, and one or more of the image sensors may perform the sensing of a plurality of types of light. Furthermore, in a case where the endoscope 121 includes a plurality of image sensors capable of performing the sensing of a plurality of types of light, at least one or more of the image sensors may perform the sensing of light of a type different from that of other image sensors.

Also, the endoscope 121 may further include a sensor other than the image sensor. For example, the endoscope 121 may further be provided with a gyro sensor which measures posture of the endoscope 121 or may include other sensors. Furthermore, the endoscope 121 may have functions and configurations other than those of the sensor.

Meanwhile, in a case where a plurality of endoscopes 121 is provided in the endoscopic surgery support system 100, not all the endoscopes 121 have to have the same specification, and the specification (configuration, function and the like) of a part of the endoscopes 121 may be different from that of at least another part of the endoscopes 121.

The energy device 122 may be electrically connected to the surgical tool control device 115 via a predetermined cable, for example, and high-frequency current and the like may be supplied from the surgical tool control device 115 via the predetermined cable. Also, the energy device 122 may be connected to the surgical tool control device 115 so as to be able to communicate with each other to exchange control information and the like. This communication may be wired communication, wireless communication, or both of them.

Meanwhile, in a case where a plurality of energy devices 122 is provided in the endoscopic surgery support system 100, it is not required that all of the energy devices 122 have the same specification, and the specification (configuration, function and the like) of a part of the energy devices 122 may be different from that of at least another part of the energy devices 122.

In a case where a plurality of pairs of forceps 123 is provided in the endoscopic surgery support system 100, it is not required that all the pairs of forceps 123 have the same specification, and the specification (size, shape, function and the like) of a part of the pairs of forceps 123 may be different from that of at least another part of the pair of forceps 123. The same also applies to the insufflation needle 124.

The monitoring sensor 125 is connected to the monitoring device 113 so as to be able to communicate with each other. This communication may be wired communication, wireless communication, or both of them.

Also, the monitoring sensor 125 may further measure a parameter other than the temperature distribution. This parameter may be arbitrary. For example, the monitoring sensor 125 may measure an image, light, electricity, sound, vibration, acceleration, a speed, an angular speed, force, temperature (not temperature distribution), humidity, a flow rate, magnetism, a chemical substance, odor and the like. Also, the monitoring sensor 125 may measure a plurality of parameters among them. Also, the monitoring sensor 125 may measure a parameter other than them. Meanwhile, one monitoring sensor 125 may measure a plurality of parameters or a plurality of monitoring sensors 125 may be provided. For example, one monitoring sensor 125 may be provided with both a thermo sensor for measuring the temperature distribution and another sensor for measuring other parameters. Also, the thermo sensor for measuring the temperature distribution and another sensor for measuring the parameter other than this may be provided as monitoring sensors 125 different from each other.

That is, in a case where a plurality of monitoring sensors 125 are provided in the endoscopic surgery support system 100, as long as the temperature distribution may be measured by any one of them, the configuration and the like other than this is arbitrary. For example, the specifications of the plurality of monitoring sensors 125 may be unified or not unified.

Also, in FIG. 1, one CCU 111, one light source device 112, one monitoring device 113, one output device 114, one surgical tool control device 115, and one insufflation device 116 are illustrated, but this configuration example is only an example and the configuration of the endoscopic surgery support system 100 is not limited to this example. For example, a plurality of at least a part of the CCU 111, the light source device 112, the monitoring device 113, the output device 114, the surgical tool control device 115, and the insufflation device 116 may be provided. In that case, the specifications of the plurality of devices provided may be unified or not unified.

The number of (corresponding) endoscopes 121 controlled by one CCU 111 to obtain the captured image may be one or two or more. Also, in a case where a plurality of CCUs 111 is provided, the CCUs 111 may correspond to the different endoscopes 121, respectively, or at least a part of them may overlap. Also, the number of the endoscopes 121 corresponding to each CCU 111 may be the same, or the number of the endoscopes 121 corresponding to a part of the CCUs 111 may be different from the number of the endoscopes 121 corresponding to at least another part of the CCUs 111.

The light source device 112 may emit light of an arbitrary frequency band. For example, the light source device 112 may emit ordinary light such as visible light, or may emit special light (for example, narrow-band light, infrared light and the like) which may especially identify the affected site. Also, one light source device 112 may supply light (correspond) to a plurality of endoscopes 121. In this case, light of the same frequency band may be supplied to each endoscope 121, or light of different frequency bands may be supplied to each endoscope 121.

Also, in a case where the endoscopic surgery support system 100 is provided with a plurality of light source devices 112, the endoscopes 121 corresponding to the light source devices 112, respectively, may be different from each other, or at least a part of them may overlap with each other. Also, the number of the endoscopes 121 corresponding to each light source device 112 may be the same, or the number of the endoscopes 121 corresponding to a part of the light source devices 112 may be different from the number of the endoscopes 121 corresponding to at least another part of the light source device 112.

Also, one monitoring device 113 may obtain information from a plurality of monitoring sensors 125 (correspond thereto). Also, in a case where the endoscopic surgery support system 100 is provided with a plurality of monitoring devices 113, the monitoring sensor 125 corresponding to each monitoring device 113 may be different from each other, or at least a part of them may overlap with each other. Also, the number of the monitoring sensors 125 corresponding to each monitoring device 113 may be the same, or the number of the monitoring sensors 125 corresponding to a part of the monitoring devices 113 may be different from the number of the monitoring sensors 125 corresponding to at least another part of the monitoring devices 113.

The output device 114 is connected to the monitoring device 113 so as to be able to communicate with each other. This communication may be wired communication, wireless communication, or both of them.

Also, one output device 114 may obtain the information from a plurality of monitoring devices 113 (correspond thereto). Also, in a case where the endoscopic surgery support system 100 is provided with a plurality of output devices 114, the monitoring devices 113 corresponding to respective output devices 114 may be different from each other, or at least a part of them may overlap with each other. Also, the number of the monitoring devices 113 corresponding to each output device 114 may be the same, or the number of the monitoring devices 113 corresponding to a part of the output devices 114 may be different from the number of the monitoring devices 113 corresponding to at least another part of the output devices 114.

Also, one surgical tool control device 115 may control (correspond to) driving of a plurality of energy devices 122. Also, in a case where the endoscopic surgery support system 100 is provided with a plurality of surgical tool control devices 115, the energy devices 122 corresponding to the respective surgical tool control devices 115 may be different from each other, or at least a part of them may overlap with each other. Also, the number of the energy devices 122 corresponding to each surgical tool control device 115 may be the same, or the number of the energy devices 122 corresponding to a part of the surgical tool control devices 115 may be different from the number of the energy devices 122 corresponding to at least another part of the surgical tool control devices 115.

Also, one insufflation device 116 may supply gas (correspond to) a plurality of insufflation needles 124. Also, in a case where the endoscopic surgery support system 100 is provided with a plurality of insufflation devices 116, the insufflation needles 124 corresponding to the respective insufflation devices 116 may be different from each other, or at least a part of them may overlap with each other. Also, the number of the insufflation needles 124 corresponding to each insufflation device 116 may be the same, or the number of the insufflation needles 124 corresponding to a part of the insufflation devices 116 may be different from the number of the insufflation needles 124 corresponding to at least another part of the insufflation devices 116.

<Range of Sensing>

Next, a range of sensing is described. The monitoring sensor 125 makes a predetermined area (around an installation position, for example) according to the installation position and posture of the monitoring sensor 125 the sensing area. The installation position of the monitoring sensor 125 is arbitrary. For example, there is a high possibility of occurrence of contact between the surgical tool and the living body (the organ and the like), bleeding and the like in the surgical site in which the surgeon performs operation (for example, the affected site to be operated) and around the same, and the necessity of monitoring is higher than that in other positions. Therefore, such surgical site and around the surgical site may be made the sensing area. That is, the monitoring sensor 125 is installed in such a position and posture that the surgical site and around the surgical site is made the sensing area. For example, as illustrated in FIG. 1, the monitoring sensor 125 is installed in a state of being inserted into the living body of the patient 151.

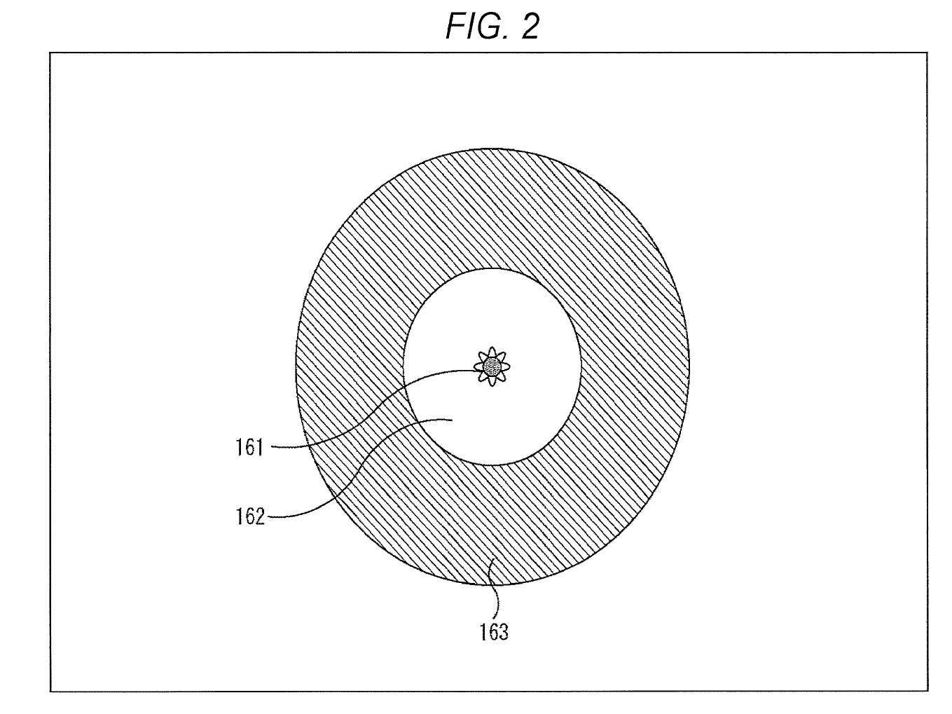

For example, in a case of FIG. 2, an affected site 161 being the surgical site and around the same are made an imaging area 162 being a range imaged by the image sensor of the endoscope 121, and a range including the imaging area 162 is further made a sensing area 163 of the monitoring sensor 125. Meanwhile, in FIG. 2, outer peripheries of the imaging area 162 and the sensing area 163 are illustrated to have circular shapes, but the shapes of the areas are arbitrary and are not limited to the example in FIG. 2.

Also, in FIG. 2, the sensing area 163 is illustrated to have a planar shape, but the sensing area 163 may also be a three or higher-dimensional space. For example, in a case of the laparoscopic surgery, a space around the surgical site in the living body of the patient may be made the sensing area. For example, by performing the sensing from a plurality of positions and directions using a plurality of monitoring sensors 125, the monitoring device 113 may obtain the temperature distribution in the three or higher-dimensional space. By doing so, it is possible to reduce a blind angle by the organ and the like and to obtain more accurate temperature distribution.

Meanwhile, it is also possible that the imaging area 162 and the sensing area 163 do not overlap with each other, or are partially overlapped. It is also possible that the affected site 161 is not included in the sensing area 163 or the affected site 161 is not included in the imaging area 162.

Generally, since resolution of the temperature distribution image may be lower than that of the captured image, as in the example in FIG. 2, the sensing area 163 may be set to be wider than the imaging area 162. However, sizes of the imaging area 162 and the sensing area 163 are arbitrary.

<Main Configuration Example of Monitoring Device and the Like>

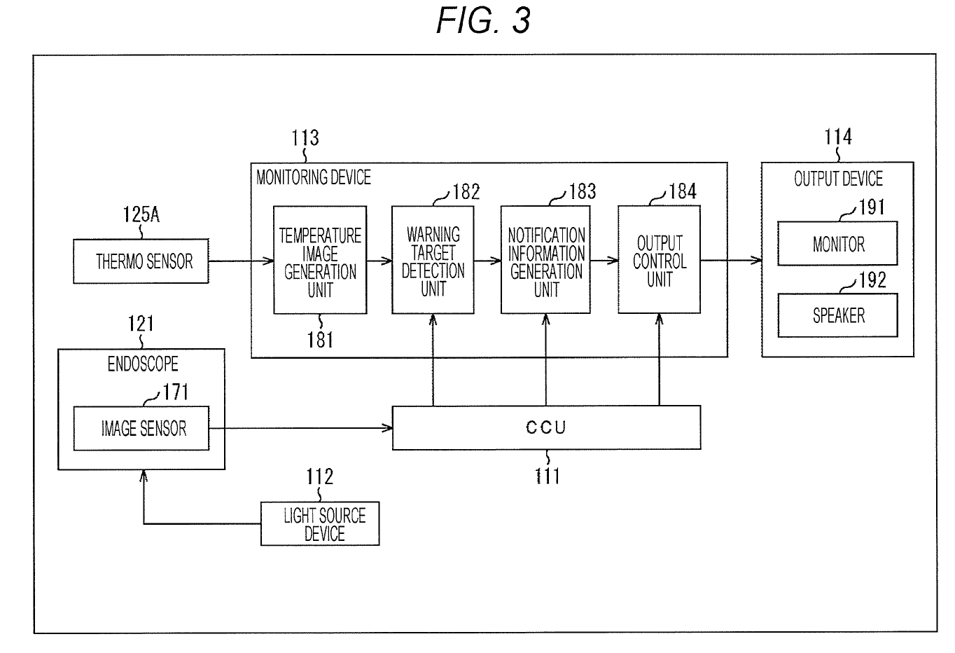

FIG. 3 is a block diagram illustrating a main configuration example of the monitoring device 113 and the like. In the example in FIG. 3, the endoscope 121 includes an image sensor 171.

The image sensor 171 includes a so-called imaging element and includes a plurality of pixels for performing photoelectric conversion of incident light. The endoscope 121 irradiates a light irradiation area with light supplied from the light source device 112. The image sensor 171 photoelectrically converts the irradiation light reflected by a subject in a plurality of pixels to obtain an image of the subject (a captured image of the imaging area). That is, the light irradiation area and the imaging area are the areas in substantially the same position. It goes without saying that the size and shape of the imaging area and those of the light irradiation area need not be the same. In this manner, the image sensor 171 images the subject and obtains an image signal of the image of the subject (captured image data). Information regarding the captured image obtained by the image sensor 171 is supplied to the CCU 111 by communication between the endoscope 121 and the CCU 111. The image sensor 171 is controlled by the CCU 111 to drive to perform such imaging.

The CCU 111 performs predetermined signal processing on the captured image data supplied from the image sensor 171 as necessary and supplies the same to the monitoring device 113 (a warning target detection unit 182, a notification information generation unit 183, and an output control unit 184).

Also, as illustrated in FIG. 3, a thermo sensor 125A is provided as the monitoring sensor 125.

The thermo sensor 125A measures the change in time of the temperature (temperature distribution) in the sensing area. An installation position of the thermo sensor 125A being the monitoring sensor 125 is arbitrary as described above. For example, the thermo sensor 125A is installed in such a position and posture that the surgical site and around the surgical site is made the sensing area. For example, as illustrated in FIG. 1, the thermo sensor 125A is installed in a state of being inserted into the living body of the patient 151, and performs the sensing of the temperature (temperature distribution) of the sensing area 163 in FIG. 2. Meanwhile, the temperature (temperature distribution) may be an absolute value or a relative value with respect to a predetermined criterion. The information regarding the sensing result obtained by the thermo sensor 125A is supplied to the monitoring device 113.

For example, the thermo sensor 125A may be controlled by an external device (for example, the monitoring device 113) to drive, or may be controlled by the surgeon and the like to drive, or may drive without being controlled by them.

In the example in FIG. 3, the monitoring device 113 includes a temperature image generation unit 181, the warning target detection unit 182, the notification information generation unit 183, and the output control unit 184.

The temperature image generation unit 181 obtains the information regarding the sensing result from the thermo sensor 125A and generates a temperature image on the basis of the information. The temperature image is an image illustrating temperature (or temperature distribution) of the sensing area. The temperature image generation unit 181 supplies the generated temperature image to the warning target detection unit 182. The thermo sensor 125A performs the sensing for a predetermined period, and the temperature image generation unit 181 generates the temperature image as a moving image of the predetermined period. That is, the temperature image generated by the temperature image generation unit 181 illustrates the change in time of the temperature (or temperature distribution) during the predetermined period.

The warning target detection unit 182 detects a predetermined event to be warned on the basis of the temperature image (that is, the change in time of the temperature (or temperature distribution) in a predetermined period). This event to be warned is set in advance. That is, a pattern of the change of the temperature (or temperature distribution) is set in advance. The warning target detection unit 182 detects the pattern of the change of the temperature (or temperature distribution) set in advance in the temperature image, thereby detecting the event corresponding to the pattern. The warning target detection unit 182 notifies the notification information generation unit 183 of the detected event. Meanwhile, the warning target detection unit 182 may also refer to the captured image supplied via the CCU 111 when detecting such event.

The notification information generation unit 183 generates the notification information for performing notification according to the event detected by the warning target detection unit 182. For example, the notification information generation unit 183 generates a warning image being image information for warning about the detected event as the notification information. Also, for example, the notification information generation unit 183 generates warning sound being sound information for warning about the detected event as the notification information. The notification information generation unit 183 supplies the notification information generated in this manner to the output control unit 184. Meanwhile, the notification information generation unit 183 may also generate the notification information with reference to the captured image supplied via the CCU 111. For example, the notification information generation unit 183 may set a layout of the warning image in accordance with a picture of the captured image.

The output control unit 184 controls an output of information. For example, the output control unit 184 generates a display image (that is, the image of the imaging area) from the captured image supplied from the CCU 111 and supplies the display image to the output device 114 to display. Also, for example, the output control unit 184 supplies the notification information (image, sound and the like) supplied from the notification information generation unit 183 to the output device 114 to output. At that time, the output control unit 184 may also superimpose the warning image supplied from the notification information generation unit 183 on the captured image supplied from the CCU 111 to generate the display image and supply the display image to the output device 114 to display. Meanwhile, the output control unit 184 may be configured to be able to supply various types of information to devices other than the output device 114.

The output device 114 includes a monitor 191 and a speaker 192. For example, the monitor 191 displays the display image and the like supplied from the output control unit 184. The speaker 192 outputs the warning sound and the like supplied from the output control unit 184. A configuration of the output device 114 is arbitrary. For example, the monitor 191 or the speaker 192 may be omitted. Also, for example, the output device 114 may be provided with a plurality of monitors 191 or a plurality of speakers 192. Furthermore, for example, the output device 114 may be provided with an output device other than the monitor 191 and the speaker 192.

As described above, since the monitoring device 113 includes the warning target detection unit 182 and the notification information generation unit 183, this may notify of more various events occurring in the surgical site or around the surgical site. Therefore, the monitoring device 113 may perform more various medical supports.

<Flow of Warning Process>

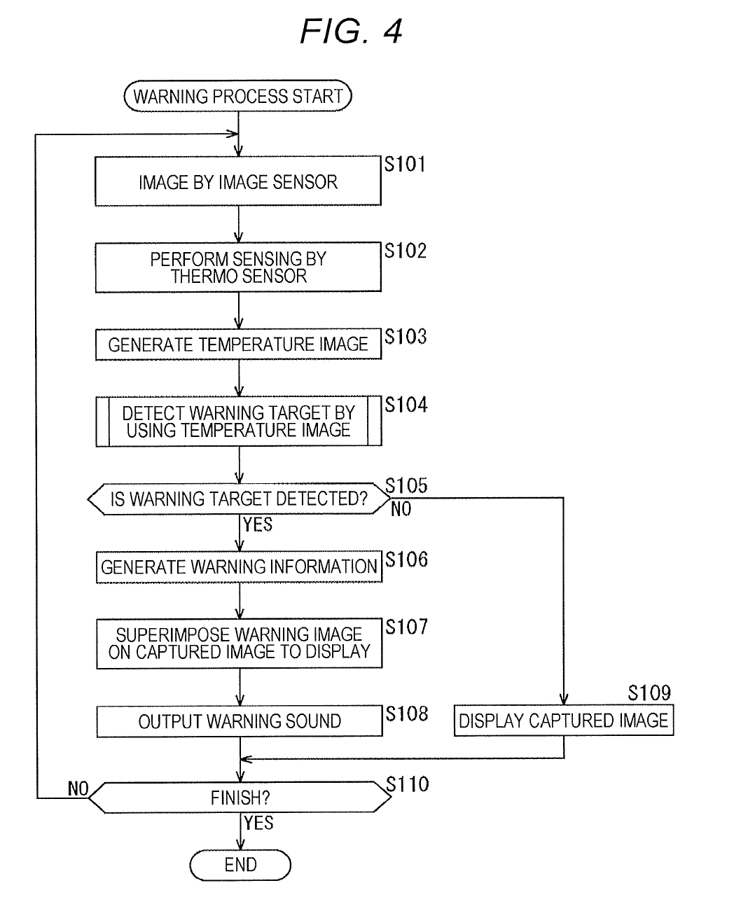

Next, with reference to a flowchart in FIG. 4, an example of a flow of a warning process executed by the endoscopic surgery support system 100 as described above is described. The warning process is a process of detecting a predetermined event to be warned on the basis of the change in time of the temperature (or temperature distribution) of the sensing area obtained as the result of the sensing by the thermo sensor 125A and generating the notification information for notifying of the event to output.

When the warning process is started, at step S101, the image sensor 171 of the endoscope 121 images a field of view of the endoscope 121 (that is, the imaging area 162). More specifically, the light source device 112 emits light (illumination light) emitted from the endoscope 121 to supply to the endoscope 121. As a result, the light is emitted from the endoscope 121 toward the inside of an angle of view of imaging (subject). In this state, the CCU 111 controls the image sensor 171 to perform the imaging. The image sensor 171 supplies the data of the captured image to the CCU 111.

At step S102, the thermo sensor 125A performs the sensing to obtain the information of the temperature (temperature distribution) of the sensing area. The thermo sensor 125A supplies the sensing result to the temperature image generation unit 181.

At step S103, the temperature image generation unit 181 generates the temperature image of the sensing area on the basis of the sensing result of the thermo sensor 125A obtained by the process at step S102.

At step S104, the warning target detection unit 182 detects the warning target by using the temperature image obtained by the process at step S103.

At step S105, the warning target detection unit 182 determines whether the warning target is detected. In a case where it is determined that the warning target is detected, the procedure shifts to step S106.

At step S106, the notification information generation unit 183 generates the warning information for warning about the event to be warned as the notification information. The warning information includes the warning image being the image information, the warning sound being the sound information and the like.

At step S107, the output control unit 184 superimposes the warning image generated by the process at step S106 on the captured image obtained by the process at step S101 supplied from the CCU 111 to generate the display image and displays the same on the monitor 191 of the output device 114.

At step S108, the output control unit 184 outputs the warning sound generated by the process at step S106 from the speaker 192 of the output device 114.

Meanwhile, in a case where it is determined at step S105 that the warning target is not detected, at step S109, the output control unit 184 generates the display image displaying the captured image obtained by the process at step S101 supplied from the CCU 111 and displays the same on the monitor 191 of the output device 114.

At step S110, the output control unit 184 determines whether to finish the warning process. In a case where it is determined that the warning process is not finished, the procedure returns to step S101 and the subsequent processes are repeated. That is, while the warning process is being executed, the processes at steps S101 to S110 are executed. Then, in a case where it is determined at step S110 that the warning process is finished, the warning process is finished.

While the warning process is being executed, the processes at steps S101 to S110 are executed in parallel. That is, the processes such as the generation of the temperature image, the detection of the warning target, the generation of the notification information, the output of the notification information and the like are performed while the image sensor 171 images and the thermo sensor 125A performs the sensing.

Therefore, the temperature image as the moving image is obtained by the warning target detection unit 182. Therefore, the warning target detection unit 182 detects the pattern of the change in time of the temperature corresponding to the event to be warned in the moving image.

<Occurrence of Bleeding>

The event to be warned is arbitrary. For example, occurrence of bleeding may be made the event to be warned.

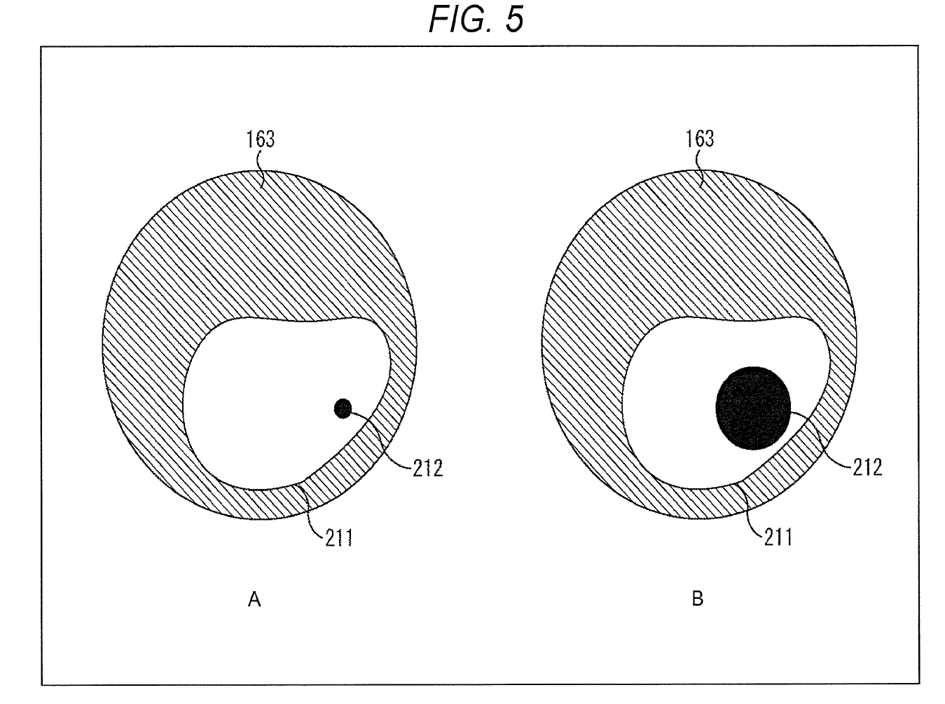

Generally, temperature of the blood that is freshly bleeding is higher than temperature outside the organ (deep body temperature). Therefore, in the temperature image, temperature of a portion of the bleeding blood is illustrated to be higher than that of its surroundings. FIG. 5 illustrates an example of the temperature image. A of FIG. 5 illustrates the example of the temperature image at certain time. In a case of the example in A of FIG. 5, an area 212 at temperature higher than that of its surroundings is detected in an organ 211 located in the sensing area 163.

B of FIG. 5 illustrates the example of the temperature image predetermined time after a state of A of FIG. 5. As illustrated in B of FIG. 5, at this time point, the area 212 enlarges from that in the time point of A of FIG. 5. That is, as time elapses, the area 212 at temperature higher than that of its surroundings enlarges.

In a case where such change in time is detected in the temperature image, the warning target detection unit 182 determines that this area 212 is the bleeding blood and detects the occurrence of the bleeding.

<Flow of Bleeding Detection Process>

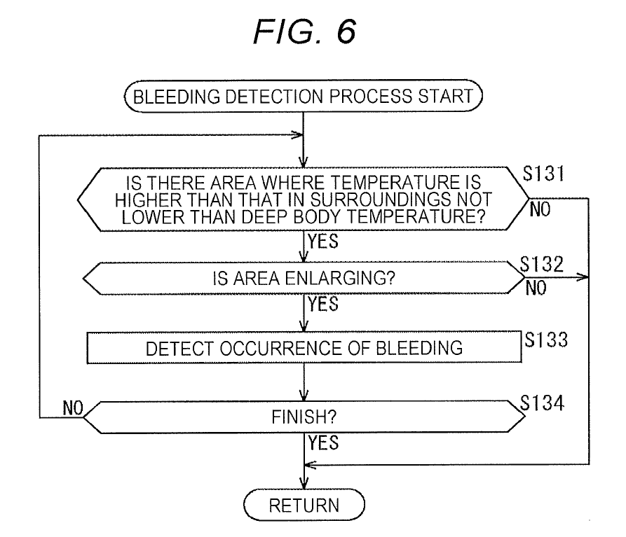

In a case of detecting the occurrence of the bleeding, the warning target detection unit 182 performs such a process as an event detection process at step S104 of the warning process. An example of a flow of a bleeding detection process is described with reference to a flowchart in FIG. 6.

When the bleeding detection process is started, at step S131, the warning target detection unit 182 determines whether an area at temperature higher than that of its surroundings not lower than the deep body temperature occurs in the temperature image. In a case where it is determined that the area at the temperature higher than that of its surroundings not lower than the deep body temperature such as the area 212 in A of FIG. 5 is generated, the procedure shifts to step S132.

At step S132, the warning target detection unit 182 determines whether the area enlarges with the lapse of time. In a case where it is determined that the area enlarges with the lapse of time in the temperature image as the area 212 in B of FIG. 5, the procedure shifts to step S133.

At step S133, the warning target detection unit 182 determines that the area is the bleeding blood and detects the occurrence of the bleeding.

At step S134, the warning target detection unit 182 determines whether to finish the bleeding detection process. In a case where it is determined not to finish, the procedure returns to step S131 and the subsequent processes are repeated.

Meanwhile, in a case where it is determined at step S131 that no area at the temperature higher than that of its surroundings not lower than the deep body temperature is generated in the temperature image, the bleeding detection process is finished and the procedure returns to FIG. 4. Also, in a case where it is determined at step S132 that the area at the temperature higher than that of its surroundings not lower than the deep body temperature does not enlarge, the warning target detection unit 182 determines that the area is not the bleeding blood, the bleeding detection process is finished, and the procedure returns to FIG. 4. Also, in a case where it is determined to finish at step S134, the bleeding detection process is finished and the procedure returns to FIG. 4.

By performing each process as described above, the monitoring device 113 may detect the occurrence of the bleeding by using the change in time of the temperature (temperature distribution). Therefore, the monitoring device 113 may detect more various events as compared to a case of detecting the occurrence of the bleeding from simple temperature distribution (temperature distribution at certain time). In addition, the monitoring device 113 may more accurately detect the occurrence of the bleeding as compared to a case of detecting the occurrence of the bleeding from the simple temperature distribution (temperature distribution at certain time).

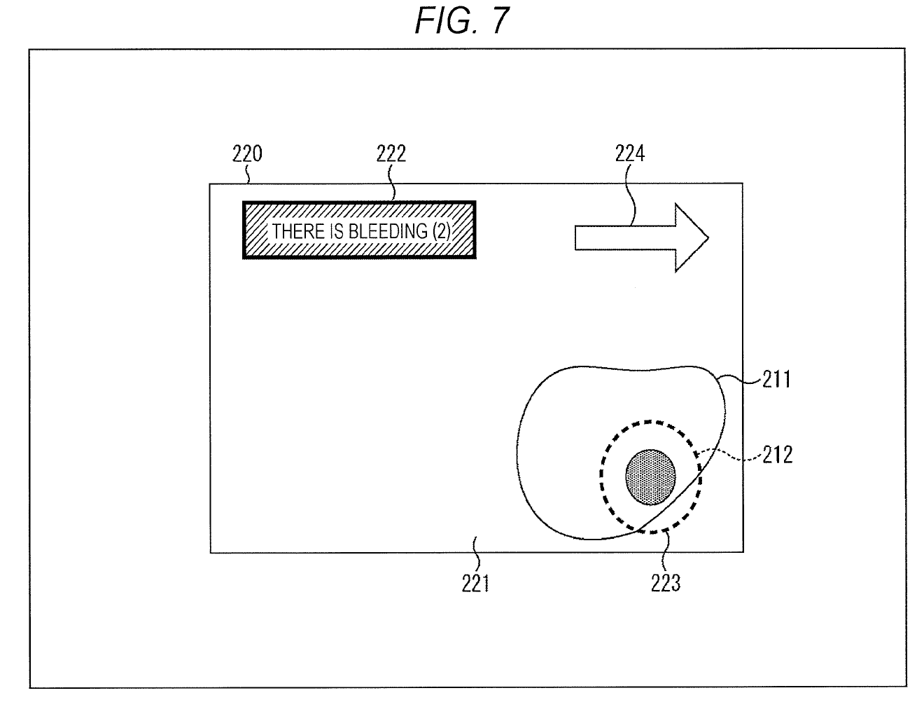

FIG. 7 illustrates an example of the display image displayed on the monitor 191. A display image 220 illustrated in FIG. 7 illustrates an example of the display image including the warning image in a case where the occurrence of the bleeding is detected by performing the bleeding detection process described above. As illustrated in FIG. 7, in the display image 220, various warning images are superimposed on a captured image 221 including the organ 211.

A bleeding warning message image 222 being an image notifying of the occurrence of the bleeding may be displayed as the warning image as in the display image 220 illustrated in FIG. 7. The bleeding warning message image 222 in FIG. 7 includes a message “there is bleeding” and a number “(2)”. The message “there is bleeding” is the message notifying of the occurrence of the bleeding. As a result, the surgeon and the like who looks at the display image 220 may grasp the occurrence of the bleeding more easily. Also, the number “(2)” indicates the number of sites where the bleeding occurs. Therefore, the surgeon and the like who looks at the display image 220 may more easily grasp the number of sites where the bleeding occurs.

That is, the notification information generation unit 183 may generate such bleeding warning message image 222 as the warning image. Meanwhile, a display position of the bleeding warning message image 222 is arbitrary, and it is not necessary to be on upper left of the display image as in the example in FIG. 7. The notification information generation unit 183 may set the display position of the bleeding warning message image 222 on the basis of a picture of the captured image 221.

Also, in a case where the site where the bleeding occurs is located in the captured image 221 as in the display image 220 illustrated in FIG. 7, a bleeding position presentation image 223 being an image illustrating the occurrence site may be displayed as the warning image. The bleeding position presentation image 223 in FIG. 7 is an image of a dotted circle. In the display image 220, a bleeding occurrence site (area 212) is enclosed by this dotted circle to illustrate the site where the bleeding occurs. As a result, the surgeon and the like who looks at the display image 220 may grasp more easily the site where the bleeding occurs.

That is, the notification information generation unit 183 may generate such bleeding position presentation image 223 as the warning image. Meanwhile, in a case where the sensing area of the thermo sensor 125A and the imaging area of the image sensor 171 do not coincide with each other, it is not possible to directly apply the bleeding occurrence position in the sensing area to the captured image 221. In such a case, the notification information generation unit 183 specifies the position of the site where the bleeding occurs in the captured image 221 on the basis of both the captured image 221 and the temperature image. That is, a display position of the bleeding position presentation image 223 in the display image 220 is set.

Meanwhile, in a case where the bleeding occurrence site is located outside the captured image 221, a bleeding direction presentation image 224 being an image illustrating a direction of the occurrence site may be displayed as the warning image. The bleeding direction presentation image 224 in FIG. 7 is an image of an arrow. That is, the direction of the bleeding occurrence site located outside the display image 220 is indicated by the arrow. As a result, the surgeon and the like who looks at the display image 220 may grasp the direction in which the bleeding occurs more easily.

That is, the notification information generation unit 183 may generate such bleeding direction presentation image 224 as the warning image. Meanwhile, in a case where the sensing area of the thermo sensor 125A does not coincide with the imaging area of the image sensor 171, the bleeding occurrence position in the sensing area as viewed from the captured image 221 is not specified. In such a case, the notification information generation unit 183 specifies a positional relationship between the captured image and the temperature image on the basis of both the captured image 221 and the temperature image and specifies the direction of the site where the bleeding occurs in the captured image 221. That is, the direction of the arrow of this bleeding direction presentation image 224 in the display image 220 is set. Meanwhile, a display position of the bleeding direction presentation image 224 is arbitrary, and it is not necessary to be on upper right of the display image as in the example in FIG. 7. The display position may be determined according to the direction of the arrow or the notification information generation unit 183 may set the display position on the basis of the picture of the captured image 221.

Meanwhile, contents of the warning image generated by the notification information generation unit 183 are arbitrary. The notification information generation unit 183 may generate as the warning image one or more of the image notifying of the occurrence of the bleeding, the image notifying of the number of times of occurrence of the bleeding, the image illustrating the occurrence site in a case where the bleeding occurrence site is located within the captured image range, and the image illustrating the direction of the occurrence site in a case where the bleeding occurrence site is located outside the captured image range described above. Furthermore, the notification information generation unit 183 may include other information in the warning image.

By executing each process as described above, the monitoring device 113 may notify of the event detected using the change in time of the temperature (temperature distribution). That is, the monitoring device 113 makes it possible to notify of more various events.

Meanwhile, while the surgical tool such as the energy device 122 is being used, a possibility of the occurrence of such bleeding increases. Therefore, the temperature of the surgical tool such as the energy device 122, a use condition (switch on/off) and the like may also be detected to be utilized for determining the bleeding. That is, for example, it is also possible that the bleeding is determined only in a case where the energy device 122 is being used and where the temperature in the vicinity of a distal end of the energy device 122 is high. In this manner, by adding a condition such as likelihood of the bleeding to a determination criterion of the bleeding, the monitoring device 113 may more accurately detect the occurrence of the bleeding.

Also, for example, in a case where the energy device 122 is being used, or where the temperature in the vicinity of the distal end of the energy device 122 is high, it is possible to weight the determination by using the condition such as the likelihood of the bleeding, for example, by making the bleeding be easily determined. By doing so, the monitoring device 113 may more correctly detect the occurrence of bleeding.

### 2. Second Embodiment

<Detection of Residual Heat after Use of Surgical Tool>

Meanwhile, an event which a monitoring device 113 may detect is not limited to an example described above. For example, the monitoring device 113 may monitor temperature (residual heat) of a surgical tool such as an energy device 122 after use.

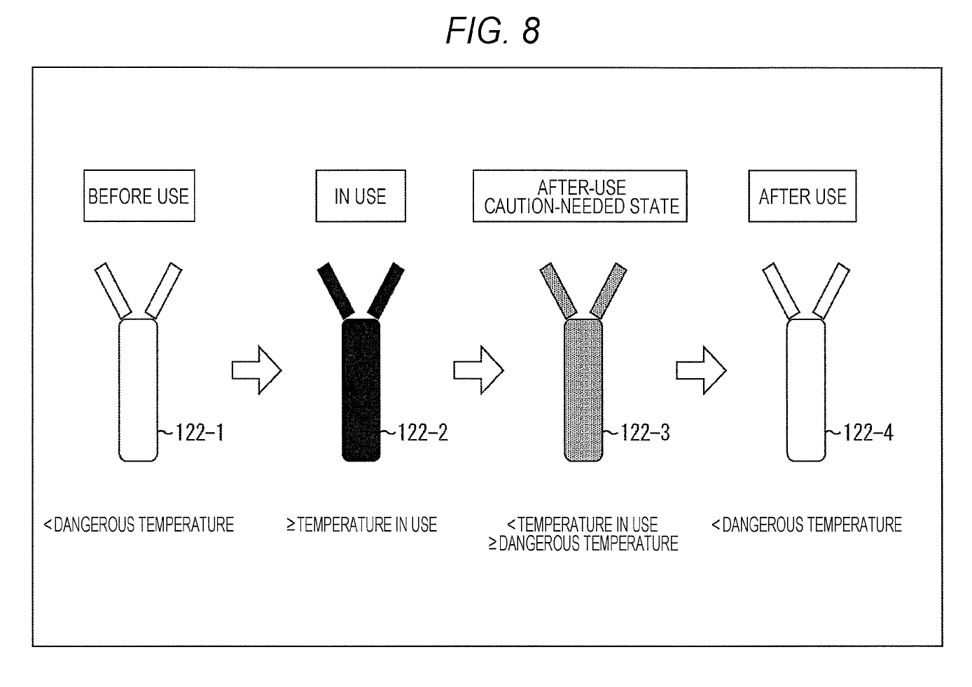

The temperature in the vicinity of a distal end of the energy device 122 changes, for example, as illustrated in FIG. 8. That is, the temperature in the vicinity of the distal end of an energy device 122-1 before use is lower than dangerous temperature being predetermined temperature which might affect a living body (organ and the like) at the time of contact. When the energy device is used, the vicinity of the distal end thereof becomes extremely hot. Therefore, the temperature in the vicinity of the distal end of an energy device 122-2 in use is not lower than predetermined temperature (temperature in use) higher than the dangerous temperature.

When the use of the energy device 122 is finished, the temperature (residual heat) in the vicinity of the distal end decreases, but the temperature is high for a while, and this is in a temperature state in which one should take care to avoid contact with the living body (organ and the like) (after-use caution-needed state). That is, the temperature in the vicinity of the distal end of an energy device 122-3 in the after-use caution-needed state is lower than the temperature in use and higher than the dangerous temperature. Thereafter, after time elapses, the temperature in the vicinity of the distal end of the energy device 122 further decreases and reaches a safe state. That is, the temperature in the vicinity of the distal end of an energy device 122-4 after use is lower than the dangerous temperature and returns to the same state as that of the energy device 122-1 before use.

In a case where the temperature in the vicinity of the distal end of the energy device 122 is higher than the dangerous temperature, if the vicinity of the distal end of the energy device 122 comes into contact with or comes close to the living body (organ and the like), this might affect the living body. That is, during use and in the after-use caution-needed state, the vicinity of the distal end of the energy device 122 is in a high temperature state which might affect the living body (organ and the like). However, since a surgeon operates the energy device 122-2 in use under intention, surgeon's attention is directed to the distal end of the energy device 122 even when it is not warned. Rather, as medical work is in progress, display of a warning image and an output of warning sound might hinder a field of view and concentration of the surgeon.

Therefore, the monitoring device 113 does not warn while being used and warns only in the after-use caution-needed state.

<Flow of after-Use Caution-Needed State Detection Process>

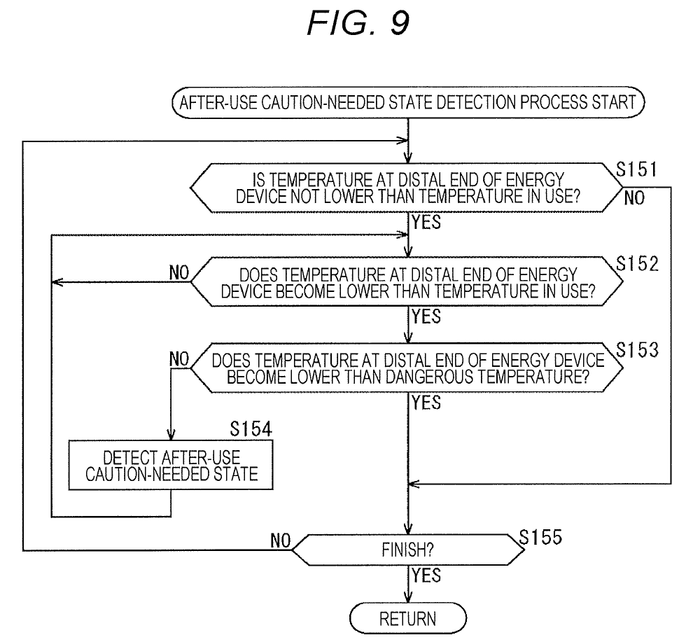

In this case, at step S104 of a warning process, a warning target detection unit 182 performs an after-use caution-needed state detection process as an event detection process. An example of a flow of the after-use caution-needed state detection process is described with reference to a flowchart in FIG. 9.

It is supposed that the after-use caution-needed state detection process starts when the energy device 122 is in a state before use (energy device 122-1) in FIG. 8. When the after-use caution-needed state detection process starts, at step S151, the warning target detection unit 182 determines whether the temperature of the distal end of the energy device 122 is not lower than the temperature in use. In a case where it is determined that the temperature is lower than the temperature in use, since the energy device 122 is in the state before use in FIG. 8, processes at step S152 to step S154 are omitted, and the procedure shifts to step S155.

Also, in a case where it is determined at step S151 that the temperature of the distal end of the energy device 122 is not lower than the temperature in use, the procedure shifts to step S152. That is, the energy device 122 is put into a state in use in FIG. 8. In this state, no warning is given as described above.

At step S152, the warning target detection unit 182 determines whether the temperature of the distal end of the energy device 122 becomes lower than the temperature in use. In a case where it is determined that the temperature of the distal end of the energy device 122 is not lower than the temperature in use, the energy device 122 is still in the state in use in FIG. 8, so that the process at step S152 is repeated.

Also, in a case where it is determined at step S152 that the temperature of the distal end of the energy device 122 becomes lower than the temperature in use, the procedure shifts to step S153. That is, the energy device 122 is put into a state after the after-use caution-needed state in FIG. 8.

At step S153, the warning target detection unit 182 determines whether the temperature of the distal end of the energy device 122 becomes lower than the dangerous temperature. In a case where it is determined that the temperature of the distal end of the energy device 122 is not lower than the dangerous temperature, the energy device 122 is in the after-use caution-needed state in FIG. 8. Therefore, at step S154, the warning target detection unit 182 detects that the energy device 122 is in the after-use caution-needed state as the warning target.

Accordingly, warning information is generated by processes at steps S106 to S108 in FIG. 4, and the image, sound and the like are output.

Returning to FIG. 9, when the process at step S154 is finished, the procedure returns to step S152, and the subsequent processes are repeated. That is, the after-use caution-needed state is detected until the temperature of the distal end of the energy device 122 becomes lower than the dangerous temperature.

In a case where it is determined at step S153 that the temperature of the distal end of the energy device 122 becomes lower than the dangerous temperature, since the energy device 122 is put into a state after use in FIG. 8, the detection of the after-use caution-needed state is finished and the procedure shifts to step S155.

At step S155, the warning target detection unit 182 determines whether to finish the after-use caution-needed state detection process. In a case where it is determined not to finish, the procedure returns to step S151 and the subsequent processes are repeated. That is, the energy device 122 returns to the state before use in FIG. 8 and each of the above-described processes are repeated.

Then, in a case where it is determined to finish at step S155, the after-use caution-needed state detection process is finished and the procedure returns to FIG. 4.

By performing each process as described above, the monitoring device 113 may detect that the surgical tool after use is in the state (after-use caution-needed state) in which the temperature is (lower than the temperature in use and) not lower than the predetermined temperature (dangerous temperature) by using change in time of the temperature (temperature distribution). Therefore, the monitoring device 113 may warn only in the after-use caution-needed state without warning while the surgical tool is used. Therefore, the monitoring device 113 may warn more appropriately without interfering with the operation of the surgeon. Since it is difficult to determine whether it is in use or in the after-use caution-needed state from simple temperature distribution (temperature distribution at certain time), it is difficult to perform such control.

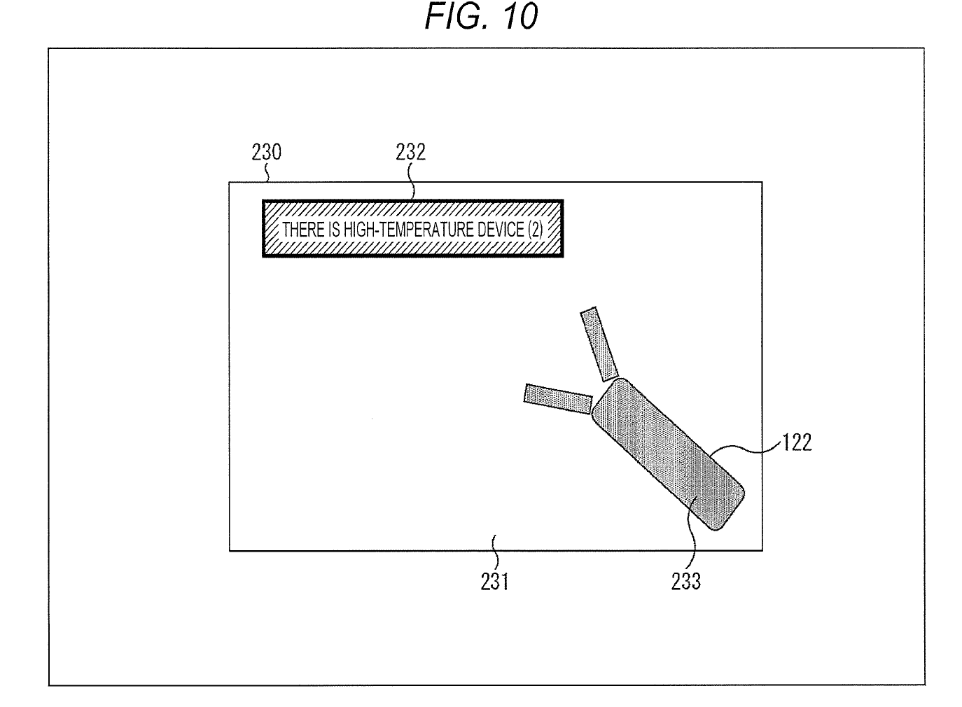

FIG. 10 illustrates an example of a display image displayed on a monitor 191 in this case. A display image 230 illustrated in FIG. 10 illustrates an example of the display image including the warning image which is displayed only in the after-use caution-needed state. As illustrated in FIG. 10, in the display image 230, various warning images are superimposed on a captured image 231 including an image in the vicinity of the distal end of the energy device 122.

As in the display image 230 illustrated in FIG. 10, as the warning image, a residual heat warning message image 232 being an image to notify of a fact that there is the surgical tool in the after-use caution-needed state (that is, there is the surgical tool after use in a state of predetermined temperature or higher) may be displayed. The residual heat warning message image 232 in FIG. 10 includes a message “there is high-temperature device” and a number “(2)”. The message “there is high-temperature device” is a message to notify of the fact that there is the surgical tool in the after-use caution-needed state. As a result, the surgeon and the like who looks at the display image 230 may more easily grasp that there is the surgical tool in the after-use caution-needed state. Also, the number “(2)” indicates the number of surgical tools that are in the after-use caution-needed state. Therefore, the surgeon and the like who looks at the display image 230 may grasp more easily the number of the surgical tools in the after-use caution-needed state.

That is, a notification information generation unit 183 may generate such residual heat warning message image 232 as the warning image. Meanwhile, a display position of the residual heat warning message image 232 is arbitrary, and it is not necessary to be on upper left of the display image as in the example in FIG. 10. The notification information generation unit 183 may set the display position of the residual heat warning message image 232 on the basis of a picture of the captured image 231.

Also, in a case where the vicinity of the distal end of the surgical tool in the after-use caution-needed state is located in the captured image 231 as the display image 230 illustrated in FIG. 10, a residual heat warning image 233 illustrating the surgical tool (energy device 122) may be displayed as the warning image. As illustrated in FIG. 10, this residual heat warning image 233 includes, for example, a temperature image in the vicinity of the distal end of this surgical tool (energy device 122). That is, the temperature image in the vicinity of the distal end of the surgical tool in the after-use caution-needed state is superimposed on the same portion of the captured image 231 (in the vicinity of the distal end of the surgical tool in the after-use caution-needed state) to be displayed. As a result, the surgeon and the like who looks at the display image 230 may intuitively grasp the temperature of the surgical tool (at least the fact that the temperature of the surgical tool which is in the after-use caution-needed state is the temperature that requires attention).

Meanwhile, the notification information generation unit 183 may easily obtain the vicinity of the distal end of the energy device 122 in the temperature image by a temperature difference with its surroundings. Also, in the captured image 231, the notification information generation unit 183 may easily obtain the vicinity of the distal end of the energy device 122 from a characteristic (picture) of the image. Also, by using both the temperature image and the captured image 231, the notification information generation unit 183 may more easily obtain the vicinity of the distal end of the energy device 122.

Meanwhile, as the residual heat warning image 233, a predetermined image for emphasizing the surgical tool may be superimposed in place of the temperature image. Meanwhile, in a case where a sensing area of a thermo sensor 125A and an imaging area of an image sensor 171 do not coincide with each other, it is not possible to directly apply a position of the surgical tool in the sensing area to the captured image 231. In such a case, the notification information generation unit 183 specifies the position of the surgical tool in the captured image 221 on the basis of both the captured image 231 and the temperature image. That is, a display position of this residual heat warning image 233 in the display image 230 is set.

Meanwhile, in a case where the vicinity of the distal end of the surgical tool in the after-use caution-needed state is located outside the captured image 231, an image illustrating a direction of the surgical tool may be displayed as the warning image. For example, the direction of the surgical tool may be indicated by an arrow. As a result, the surgeon and the like who looks at the display image 230 may more easily grasp the direction of the vicinity of the distal end of the surgical tool in the after-use caution-needed state. Meanwhile, the notification information generation unit 183 may specify the direction of the vicinity of the distal end of the surgical tool in the after-use caution-needed state on the basis of both the captured image 221 and the temperature image.

Meanwhile, contents of the warning image generated by the notification information generation unit 183 are arbitrary. The notification information generation unit 183 may generate as the warning image one or more of the image notifying of the presence of the surgical tool after use in the state of predetermined temperature or higher, the image notifying of the number of the surgical tools, the image illustrating the surgical tool in a case where the surgical tool is located within the captured image range, and the image illustrating the direction of the surgical tool in a case where the surgical tool is located outside the captured image range described above. Furthermore, the notification information generation unit 183 may include other information in the warning image.

By executing each process as described above, the monitoring device 113 may notify of the event detected using the change in time of the temperature (temperature distribution). That is, the monitoring device 113 makes it possible to notify of more various events.

Meanwhile, an operation state of the surgical tool such as the energy device 122 may also be detected from an on/off state and the like of a switch of the energy device 122 and the like. By doing so, the operating state of the energy device 122 may be detected more accurately, and the monitoring device 113 may more accurately detect the after-use caution-needed state.

Meanwhile, for example, by providing a device such as an LED on a handle of the energy device 122 and allowing the same to emit light, the above-described warning regarding the residual heat may be performed. Also, such warning may be used together with the warning by the display image 230 described above. By doing like this, it is possible to give a warning to the surgeon and the like more certainly.

<Other Warning Example>

The monitoring device 113 may also warn about events other than those described above in the manner similar to that of the warning process described above.

For example, the warning target detection unit 182 of the monitoring device 113 may detect a result (success or failure) of hemostasis as an event on the basis of the change in time such as a temperature decrease of a bleeding area or a temperature increase of a blood vessel portion in the temperature image. Then, the notification information generation unit 183 may generate, as the warning image, an image notifying of the result of the hemostasis and an image notifying of the number of sites where the hemostasis is performed, and the output control unit 184 may superimpose the warning image on the captured image to display on the monitor 191. Also, in a case where the hemostasis is performed within the range of the captured image, the notification information generation unit 183 may generate an image illustrating the site where the hemostasis is performed as the warning image, and the output control unit 184 may superimpose the warning image on the captured image and display the same on the monitor 191. Furthermore, in a case where the hemostasis is performed outside the range of the captured image, the notification information generation unit 183 may generate an image illustrating a direction in which the hemostasis is performed as the warning image, and the output control unit 184 may superimpose the warning image on the captured image and display the same on the monitor 191.

Meanwhile, contents of the warning image generated by the notification information generation unit 183 are arbitrary. The notification information generation unit 183 may generate, as the warning image, one or more of the image notifying of the result of the hemostasis, the image notifying of the number of sites where the hemostasis is performed, the image illustrating the site in which the hemostasis is performed in a case where the hemostasis is performed within the range of the captured image, and an image illustrating the direction in which the hemostasis is performed in a case where the hemostasis is performed outside the range of the captured image. Furthermore, the notification information generation unit 183 may include other information in the warning image.

Also, for example, the warning target detection unit 182 of the monitoring device 113 may detect a damaged site of the living body due to contact with the surgical tool not in use (the energy device 122 and the like) on the basis of the change in time such as the temperature increase of the living body due to inflammation and the like in the temperature image as the event. Then, the notification information generation unit 183 may generate, as the warning image, an image notifying of occurrence of the damaged site and an image notifying of the number of damaged sites, and the output control unit 184 may superimpose the warning image on the captured image to display on the monitor 191. Also, in a case where the damaged site is located within the range of the captured image, the notification information generation unit 183 may generate an image illustrating the damaged site as the warning image, and the output control unit 184 may superimpose the warning image on the captured image and display the same on the monitor 191. Furthermore, in a case where the damaged site is located outside the range of the captured image, the notification information generation unit 183 may generate an image illustrating a direction of the damaged site as the warning image, and the output control unit 184 may superimpose the warning image on the captured image to display on the monitor 191.

Meanwhile, contents of the warning image generated by the notification information generation unit 183 are arbitrary. The notification information generation unit 183 may generate as the warning image one or more of the image notifying of the occurrence of the damaged site, the image notifying of the number of the damaged sites, the image illustrating the damaged site in a case where the damaged site is located within the captured image range, and the image illustrating the direction of the damaged site in a case where the damaged site is located outside the captured image range. Furthermore, the notification information generation unit 183 may include other information in the warning image.

Also, for example, the warning target detection unit 182 of the monitoring device 113 may detect that the surgical site or around the surgical site is in a state of predetermined temperature or higher due to use of the surgical tool on the basis of the change in time such as the temperature increase of surroundings of a contact portion of the surgical tool (energy device 122 and the like) in the temperature image as the event. Then, the notification information generation unit 183 may generate, as the warning image, an image notifying of occurrence of a site in a state of predetermined temperature of higher and an image notifying of the number of sites in the state of the predetermined temperature or higher, and the output control unit 184 may superimpose the warning image on the captured image to display on the monitor 191. Also, in a case where the site in the state of the predetermined temperature or higher is located within the range of the captured image, the notification information generation unit 183 may generate an image illustrating an occurrence position of the site in the state of the predetermined temperature or higher as the warning image, and the output control unit 184 may superimpose the warning image on the captured image and display the same on the monitor 191. Furthermore, in a case where the site in the state of the predetermined temperature or higher is located outside the range of the captured image, the notification information generation unit 183 may generate an image illustrating an occurrence direction of the site in the state of the predetermined temperature or higher as the warning image and the output control unit 184 may superimpose the warning image on the captured image and display the same on the monitor 191.

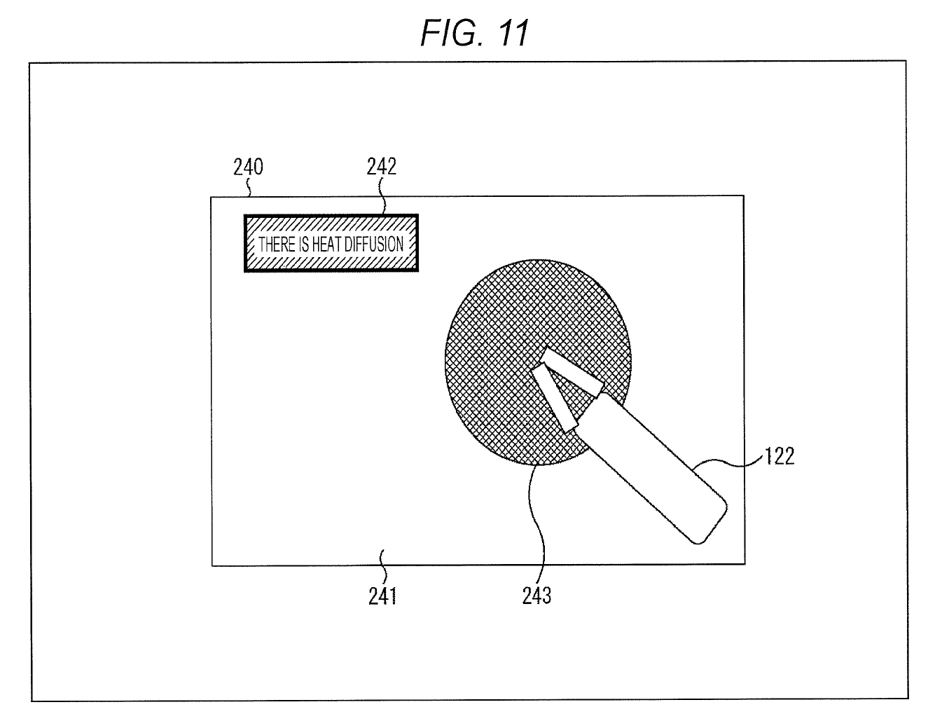

FIG. 11 illustrates an example of the display image. In a display image 240 illustrated in FIG. 11, various warning images are superimposed on a captured image 241 including an image in the vicinity of the distal end of the energy device 122. As in the display image 240 illustrated in FIG. 11, a biological temperature abnormality warning message image 242 which is an image notifying of a temperature abnormality of the living body may be displayed as the warning image. The biological temperature abnormality warning message image 242 in FIG. 11 includes a message “there is heat diffusion”. The message “there is heat diffusion” is a message notifying of occurrence of the temperature abnormality in the living body. As a result, the surgeon and the like who looks at the display image 240 may more easily grasp that the temperature abnormality of the living body occurs. Meanwhile, the biological temperature abnormality warning message image 242 may include a number indicating the number of times of occurrence of the temperature abnormality in the living body. By doing in this way, the surgeon and the like who looks at the display image 240 may more easily grasp the number of sites where the temperature of the living body or higher occurs.

That is, the notification information generation unit 183 may generate such biological temperature abnormality warning message image 242 as the warning image. Meanwhile, a display position of the biological temperature abnormality warning message image 242 is arbitrary, and it is not necessary to be on upper left of the display image as in the example in FIG. 11. The notification information generation unit 183 may set the display position of the biological temperature abnormality warning message image 242 on the basis of a picture of the captured image 241.

Also, in a case where the biological temperature abnormality is located in the captured image 241 as the display image 240 illustrated in FIG. 11, a biological temperature abnormality warning image 243 may be displayed as the image illustrating the site where the temperature abnormality occurs as the warning image. For example, a dangerous area approaching 42 degrees C. of the living body may be emphasized by a color or a pattern attached thereto such as the biological temperature abnormality warning image 243. As a result, the surgeon and the like who looks at the display image 240 may intuitively grasp where the temperature abnormality occurs.

Meanwhile, contents of the warning image generated by the notification information generation unit 183 are arbitrary. The notification information generation unit 183 may generate as the warning image, one or more of the image notifying of the occurrence of the site in the state of the predetermined or higher temperature, the image notifying of the number of sites in the state of the predetermined temperature or higher, the image illustrating the occurrence position of the site in the state of the predetermined temperature or higher in a case where the site in the state of the predetermined temperature or higher is located within the range of the captured image, and the image illustrating the occurrence direction of the site in the state of the predetermined temperature or higher in a case where the site in the state of the predetermined temperature or higher is located outside the range of the captured image. Furthermore, the notification information generation unit 183 may include other information in the warning image.

As described above, the monitoring device 113 may detect more various events on the basis of the change in time of the temperature (temperature distribution), and further notify of the various events.

### 3. Third Embodiment

<Depth Detection>

Meanwhile, the monitoring device 113 may also detect an event using information other than a temperature image. For example, it is also possible to detect a depth using a stereo image obtained by using a stereo image sensor, and detect a state in which a surgical tool and a living body are likely to be in contact with each other as an event.

<Main Configuration Example of Monitoring Device and the Like>

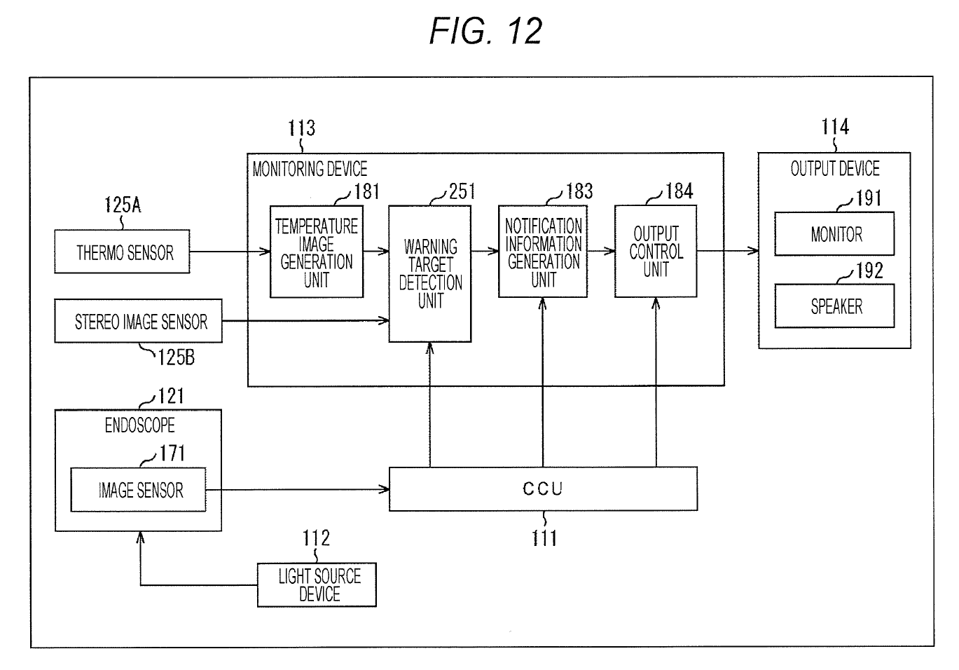

FIG. 12 is a block diagram illustrating a main configuration example of a monitoring device 113 and the like in this case. In this case also, an endoscopic surgery support system 100 basically has a configuration similar to that in a case of FIG. 3. In a case of FIG. 12, however, the endoscopic surgery support system 100 includes a stereo image sensor 125B in addition to a thermo sensor 125A as a monitoring sensor 125. Also, the monitoring device 113 includes a warning target detection unit 251 in place of a warning target detection unit 182.

The stereo image sensor 125B includes a plurality of image sensors generates the stereo image being a plurality of captured images having parallax by imaging a subject with them. The stereo image sensor 125B makes an area substantially similar to a sensing area of the thermo sensor 125A a sensing area. For example, the stereo image sensor 125B is installed in a state of being inserted into a living body of a patient 151 as illustrated in FIG. 1, and performs sensing of a sensing area 163 (a surgical site and around the surgical site) in FIG. 2 similar to the thermo sensor 125A. The stereo image sensor 125B supplies the generated stereo image to the warning target detection unit 251.

The warning target detection unit 251 obtains a temperature image from the temperature image generation unit 181. The warning target detection unit 251 detects the event in the living body to be warned by using the temperature image (change in time of temperature (temperature distribution)) and the stereo image obtained from the stereo image sensor 125B. The warning target detection unit 251 may also obtain a captured image of an image sensor 171 via a CCU 111, and the captured image may also be used for detecting the event in the living body to be warned. As in a case of the warning target detection unit 182, the warning target detection unit 251 notifies a notification information generation unit 183 of the detected event.

<Flow of Warning Process>

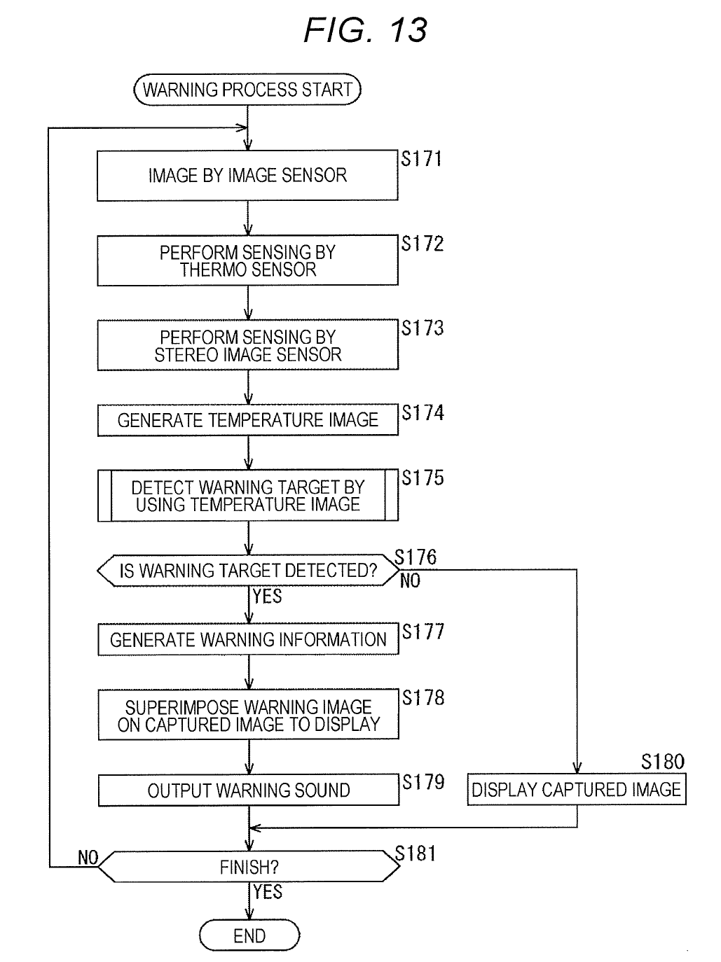

An example of a flow of a warning process in this case is described with reference to a flowchart in FIG. 13.

Processes at steps S171 and S172 are executed in a manner similar to that of processes at steps S101 and S102 in FIG. 4.

At step S173, the stereo image sensor 125B performs the sensing and generates the stereo image. The stereo image sensor 125B supplies a sensing result (stereo image) to the warning target detection unit 251.

At step S174, the temperature image generation unit 181 generates the temperature image of the sensing area on the basis of a sensing result of the thermo sensor 125A obtained by the process at step S172 as in the case at step S103 in FIG. 4.

At step S175, the warning target detection unit 251 detects the event to be warned by using the temperature image obtained by the process at step S174 and the stereo image obtained by the process at step S173.

Processes at steps S176 to S181 are executed in a manner similar to that of processes at steps S105 to S110 in FIG. 4.

In this case also, while the warning process is being executed, the processes at steps S171 to S181 are executed in parallel. That is, processes such as the generation of the temperature image, the detection of the warning target, the generation of the notification information, the output of the notification information and the like are performed while the image sensor 171 images and the thermo sensor 125A and the stereo image sensor 125B perform the sensing.

Therefore, in the warning target detection unit 251, the temperature image as a moving image is obtained. Therefore, the warning target detection unit 251 detects a pattern of the change in time of the temperature corresponding to the event in the living body to be warned in the moving image.

<Flow of after-Use Caution-Needed State Detection Process>

A case where the event in the living body is detected using the stereo image as described above is described by taking an after-use caution-needed state detection process to detect residual heat after use of the surgical tool as an example. In this case, it is warned not only that the residual heat after use of the surgical tool is not lower than dangerous temperature, but also that the surgical tool is likely to come into contact with (come close to) the living body.

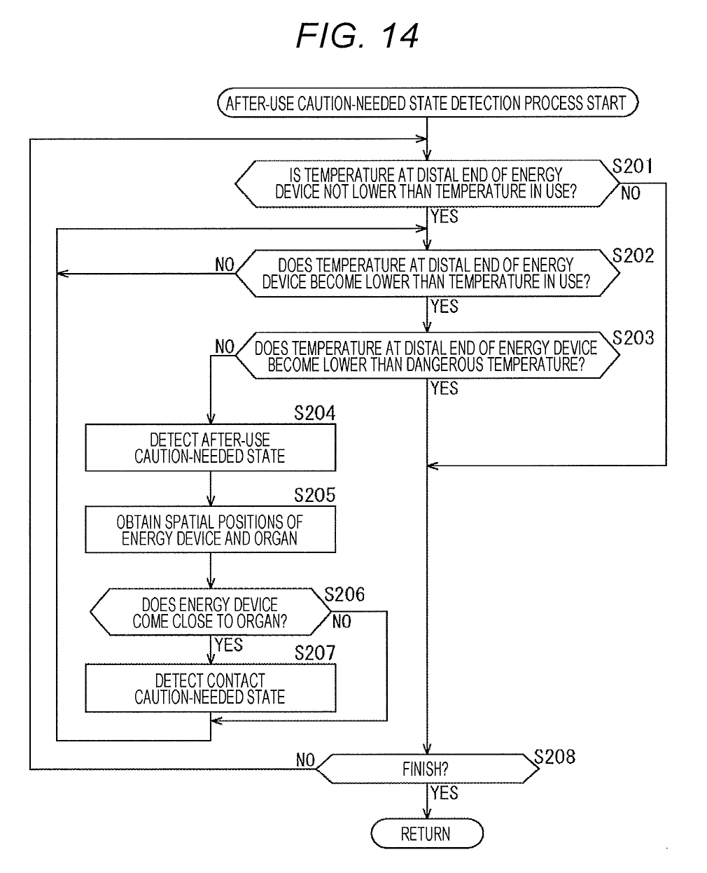

An example of a flow of the after-use caution-needed state detection process in such a case is described with reference to a flowchart in FIG. 14.

Processes at steps S201 to S204 are executed in a manner similar to that of processes at steps S151 to S154 in FIG. 9.

When an after-use caution-needed state is detected at step S204, at step S205, the warning target detection unit 251 obtains spatial positions of the surgical tool (energy device 122) and the living body (organ) by using the stereo image. For example, the warning target detection unit 251 obtains a depth position of each of the tool (energy device 122) and the living body (organ) from the stereo image.

At step S206, the warning target detection unit 251 determines whether the surgical tool (energy device 122) comes closer to the living body (organ) on the basis of the determined spatial positions.

In a case where it is determined that the surgical tool (energy device 122) comes closer to the living body (organ), at step S207, the warning target detection unit 251 detects a contact caution-needed state in which the surgical tool (energy device 122) is likely to be brought into contact with the living body (organ) as the warning target.

Therefore, in this case, warning information is generated not only for the after-use caution-needed state but also for the contact caution-needed state by processes at steps S177 to S179 in FIG. 13, and an image, sound and the like thereof are output.

Returning to FIG. 14, when the process at step S207 is finished, the procedure returns to step S202, and the subsequent processes are repeated.

Also, in a case where it is determined at step S206 that the surgical tool (energy device 122) does not come close to the living body (organ), the process at step S207 is omitted, the procedure returns to step S202, and the subsequent processes are repeated. That is, in this case, it is not in the contact attention-needed state.

Therefore, in this case, the warning information is generated only for the after-use caution-needed state by the processes at steps S177 to S179 in FIG. 13, and the image, sound and the like thereof are output.

A process at step S208 is executed in a manner similar to that of a process at step S155 in FIG. 9.

By executing each process as described above, the monitoring device 113 may warn of the state of the residual heat after use of the surgical tool using the change in time of the temperature (temperature distribution). Furthermore, the monitoring device 113 may also warn about a possibility of contact between the surgical tool and the living body on the basis of the stereo image. Meanwhile, by performing the process as described above, it is possible to warn about the contact between the surgical tool and the living body only in a case of the after-use caution-needed state in which the living body might be damaged if the surgical tool is brought into contact with the living body. That is, by inhibiting notification of unnecessary warning and by warning only in a more appropriate case, it is possible to inhibit interference with a field of view and concentration of the surgeon and the like.

### 4. Fourth Embodiment

<Diagnosis>

Meanwhile, it is also possible that a monitoring device 113 performs a process other than warning on an event in a living body detected from a temperature image and the like. For example, it is possible to evaluate the detected event in the living body and diagnose the living body. That is, it is possible to detect change in state of the living body as the event in the living body on the basis of change in time of the temperature image and generate a diagnosis image notifying of a diagnosis result based on the change in state.

For example, the monitoring device 113 may apply a stimulus (that is, heating and the like) causing the change in temperature on the living body, detect the event in the living body as the diagnosis result on the basis of a state of the change in time of temperature (temperature distribution) after that, and output diagnosis information regarding the event (diagnosis result). That is, the living body may be heated by a heating unit, and change in state of the living body after the heating may be detected.

<Main Configuration Example of Monitoring Device and the Like>

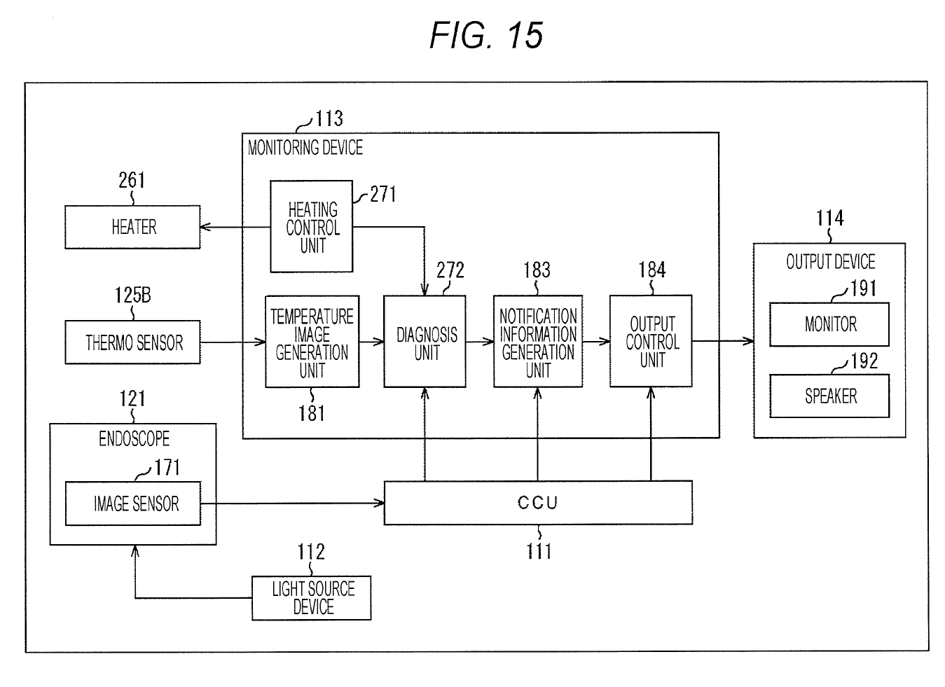

FIG. 15 is a block diagram illustrating a main configuration example of the monitoring device 113 and the like in this case. In this case also, an endoscopic surgery support system 100 basically has a configuration similar to that in a case of FIG. 3. However, in the case of FIG. 15, the endoscopic surgery support system 100 is further provided with a heater 261. Also, the monitoring device 113 is provided with a heating control unit 271 and includes a diagnosis unit 272 in place of a warning target detection unit 182 in FIG. 3.

The heater 261 is the heating unit which generates heat at predetermined temperature. The heater 261 is operated by a surgeon and the like to apply the generated heat to (to heat) the living body. A thermo sensor 125A performs sensing of a progress (change in time of the temperature) after the heating of a sensing area including a portion heated by the heater 261 of the living body.

The heating control unit 271 controls the temperature of the heat generated by the heater 261. Also, the heating control unit 271 supplies information regarding the heating to the diagnosis unit 272.

The diagnosis unit 272 diagnoses the living body and the like on the basis of the temperature image supplied from a temperature image generation unit 181. Also, the diagnosis unit 272 checks a condition of heating and the like on the basis of the information from the heating control unit 271. The diagnosis unit 272 supplies the diagnosis result to the notification information generation unit 183.

<Identification of Subsegment>

It is possible to detect a subsegment of a liver, for example, and display an area thereof by using such endoscopic surgery support system 100.

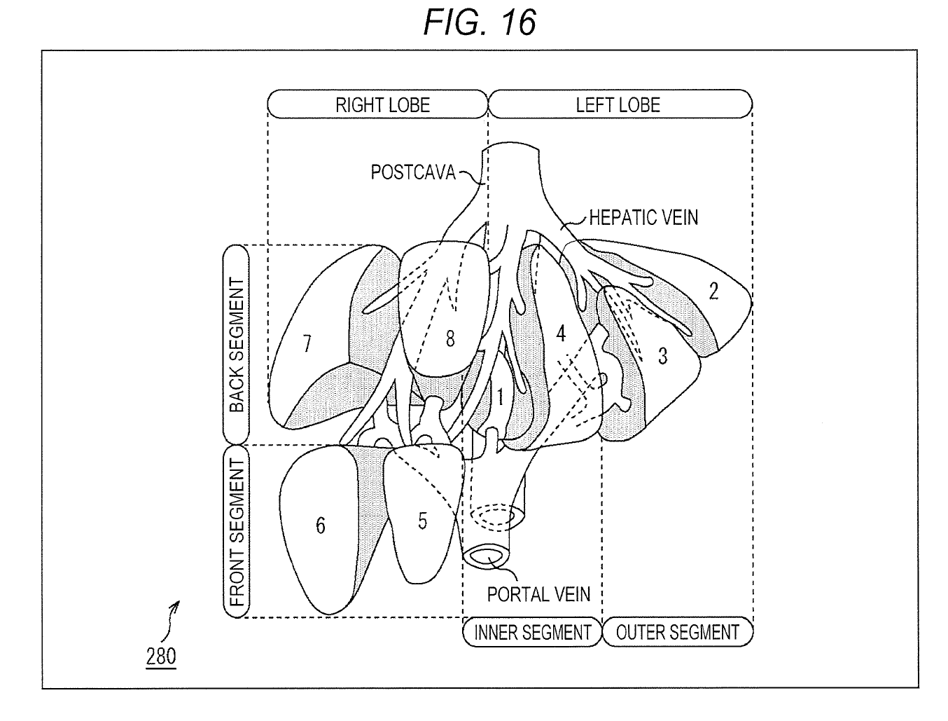

As illustrated in FIG. 16, the liver is divided into a “left lobe” and “a right lobe” on the basis of a portal vein blood flow, the left lobe further divided into an “outer segment” and an “inner segment”, and the right lobe further divided into a “front segment” and “a back segment”. Also, each of the four segments is further divided into two segments, so that eight “subsegments” are defined. Such functional division is utilized in resectional surgery and the like. For example, the “subsegment” is used as one of units of resection.

Therefore, in a case where subsegment resection of the liver is performed, for example, each subsegment is specified. When a portal vein branch (or hepatic artery branch) is heated with the heater 261 to a degree harmless to the living body, the heat propagates through blood in the portal vein branch, and temperature of a specific subsegment along the portal vein branch becomes higher than that of other subsegments. That is, the subsegment is specified by spread of an area where such a temperature increase occurs.

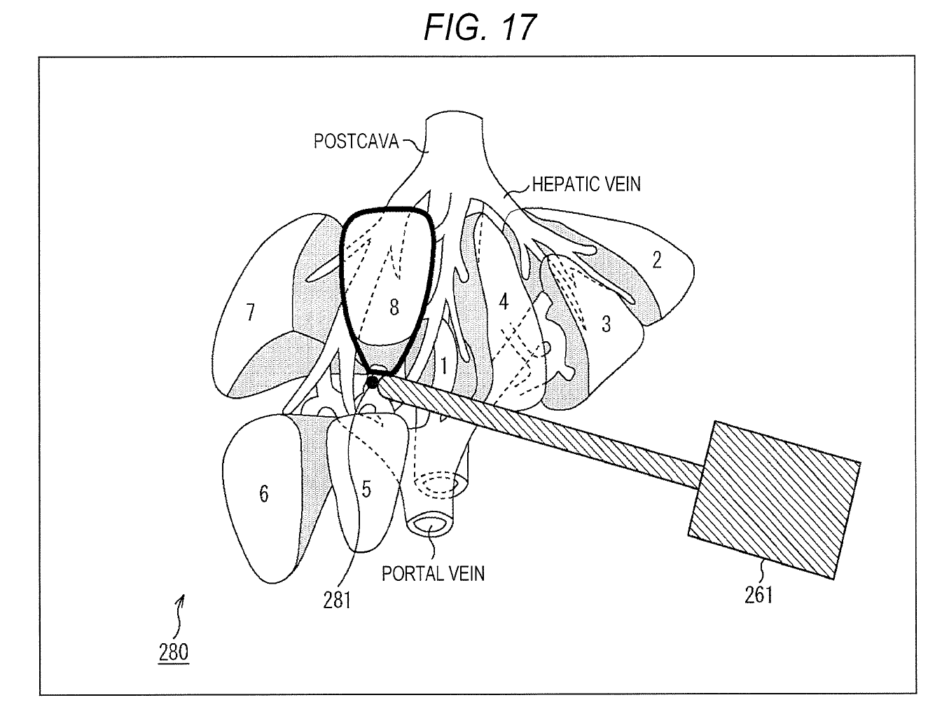

For example, as illustrated in FIG. 17, when the surgeon and the like heats a predetermined position 281 of the portal vein using the heater 261, the temperature of a subsegment “8” formed along the portal vein becomes higher than that in other subsegments. However, since other subsegments are formed along other portal vein branches, this increase in temperature is less likely to spread to other subsegments. Therefore, change in area in which the temperature increases is observed in the temperature image, and when the spread of the temperature increasing area becomes slow, the area where the temperature increases is specified as the subsegment “8”. By changing the portal vein branch to be heated, a range of another subsegment may be specified as well. That is, each subsegment may be identified.

By specifying the subsegment by such a method, it is possible to specify the subsegment more non-invasively without applying a stress on the living body of the patient as much as possible as compared to a case of using chemicals or special light.

<Flow of Diagnosis Process>

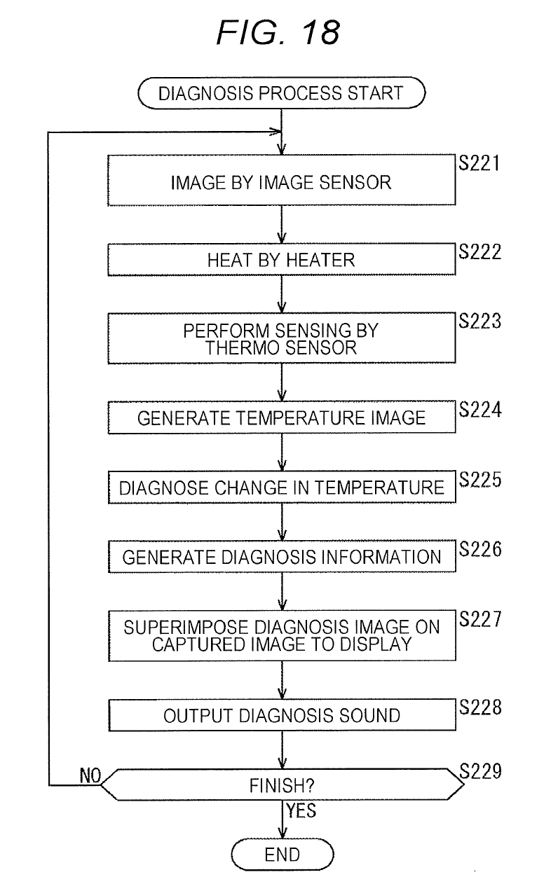

The endoscopic surgery support system 100 in this case executes a diagnosis process in place of a warning process. An example of a flow of the diagnosis process is described with reference to a flowchart in FIG. 18.

When the diagnosis process starts, at step S221, an image sensor 171 images an imaging area 162. At step S222, the heating control unit 271 controls the heater 261 to heat a predetermined portion of the living body at temperature harmless to the living body. At step S223, the thermo sensor 125A performs the sensing of the sensing area.

At step S224, the temperature image generation unit 181 generates the temperature image using the sensing result of the thermo sensor 125A.

At step S225, the diagnosis unit 272 uses the temperature image to diagnose a state of the change in time of the temperature. At step S226, the notification information generation unit 183 generates the diagnosis information indicating the diagnosis result as notification information. This diagnosis information includes, for example, a diagnosis image being an image illustrating the diagnosis result, diagnosis sound indicating the diagnosis result by sound and the like.

At step S227, the output control unit 184 superimposes the diagnosis image generated at step S226 on the captured image of the living body (surgical site) generated at step S221 and displays the same on a monitor 191.

At step S228, the output control unit 184 outputs the diagnosis sound generated at step S226 from a speaker 192.

At step S229, the output control unit 184 determines whether to finish the diagnosis process. In a case where it is determined that the diagnosis process is not finished, the procedure returns to step S221 and the subsequent processes are repeated. That is, while the diagnosis process is being executed, the processes at steps S221 to S229 are executed. Then, in a case where it is determined at step S229 that the diagnosis process is finished, the diagnosis process is finished.

While the diagnosis process is being executed, the processes at steps S221 to S229 are executed in parallel. That is, the processes such as the generation of the temperature image, the diagnosis of the change in temperature, the generation of the notification information, the output of the notification information and the like are performed while the heater 261 heats, the image sensor 171 images, and the thermo sensor 125A performs the sensing.

Therefore, in the diagnosis unit 272, the temperature image as a moving image is obtained. Therefore, the diagnosis unit 272 detects a pattern of the change in time of the temperature corresponding to the event to be diagnosed in the moving image.

<Display Image>

For example, in a case of specifying the subsegment described above, the diagnosis unit 272 observes the spread of the temperature increasing area in the temperature image, and when the spread becomes slow, this specifies the temperature increasing area as the subsegment corresponding to the heated portal vein branch. By changing the heating position, each subsegment may be specified similarly.

Then, the notification information generation unit 183 generates an image identifying each subsegment as the diagnosis image, for example, to display.

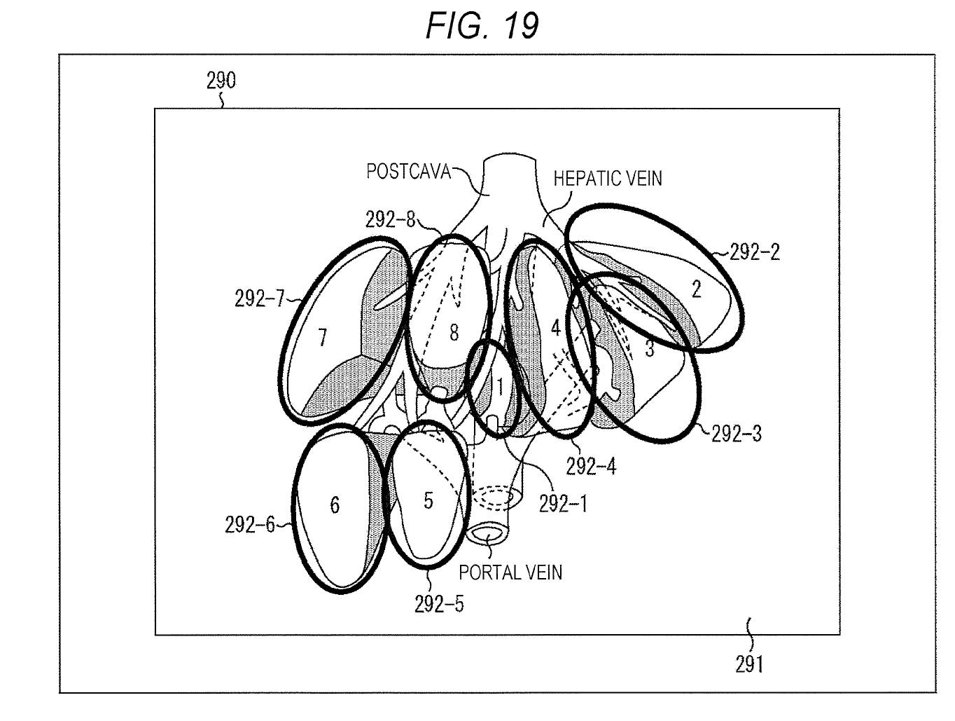

FIG. 19 is a view illustrating an example of a display image displaying the diagnosis image. A display image 290 illustrated in FIG. 19 illustrates an example of the display image including the diagnosis image in a case where the above-described diagnosis process is performed to diagnose the spread of the temperature increasing area. As illustrated in FIG. 19, in the display image 290, subsegment identification images 292-1 to 292-8 illustrating positions of the respective subsegments are superimposed on the captured image 291 of the living body (surgical site) including the liver. Meanwhile, hereinafter, in a case where it is not necessary to distinguish the subsegment identification images 292-1 to 292-8 from one another to describe, they are referred to as the subsegment identification images 292.

By displaying such subsegment identification image 292, the surgeon and the like who looks at this display image may more easily grasp the position of each subsegment. Therefore, the monitoring device 113 may make it easier for the surgeon to perform the subsegment resection and the like.

<Other Diagnosis Example>

Meanwhile, contents of the diagnosis are arbitrary and are not limited to the above-described example of the subsegment.

For example, the present technology may also be used to detect a lesion of a lung. Conventionally, a method of marking a part of the lesion with barium, a needle and the like before the surgery and grasping a part to be resected by X-ray is used. However, this method is not non-invasive, and has a large stress on the patient (living body).

Therefore, this marking is performed by the heater 261 or a similar object that generates heat (for example, a mechanism such as a portable warmer). By doing so, it is possible to easily detect the marked portion by using the thermo sensor 125A. That is, the surgeon and the like may more easily grasp the portion to be resected. Also, it is possible to reduce the stress on the patient for the marking. Furthermore, since X-ray irradiation becomes unnecessary, not only the patient but also a doctor may be prevented from exposure.

In addition, for example, the present technology may be used for blood vessel detection. In this case, for example, cold physiological saline or glucose may be administered and a temperature change event of a blood vessel may be detected from the temperature image. Also, the present technology may be used for lymphatic vessel detection. In this case, for example, cold physiological saline and the like may be administered to a lymphatic vessel and a temperature change event of the lymphatic vessel may be detected. Also, the present technology may be used to distinguish between bone and nerve. Furthermore, the present technology may be used for detecting cancer. In this case, for example, heat may be applied to the living body by the heater 261 and the like, and the cancer cell may be detected from the temperature image as an event of a difference in a manner of cooling due to a difference in thermal conductivity.

Meanwhile, the heating described above includes both heating to temperature higher than body temperature of the living body and heating to temperature lower than the body temperature (either is acceptable). Also, the state of change in time of the temperature includes both a state of a temperature increase and a state of a temperature decrease (either is acceptable).

Also, the diagnosis may be performed by selecting from a plurality of diagnosis candidates. For example, the diagnosis unit 272 may detect the subsegment of the liver, detect the lesion, detect the blood vessel, detect the lymphatic vessel, identify bone and nerve, or detect the cancer cell, and the notification information generation unit may generate an image illustrating the subsegment, the lesion, the blood vessel, the lymphatic vessel, the bone, the nerve, or the cancer cell detected by the detection unit so as to be identified which is displayed so as to be superimposed on the captured image of the living body as the diagnosis image.

### 5. Fifth Embodiment

<Control>

Meanwhile, a monitoring device 113 may perform not only notification of information such as warning but also device control of a surgical tool on the basis of an event in a living body detected from a temperature image and the like. For example, it is possible to control temperature of gas to be supplied into the living body or brightness of illumination light emitted in the living body on the basis of the temperature image measured in the living body.

<Main Configuration Example of Monitoring Device and the Like>

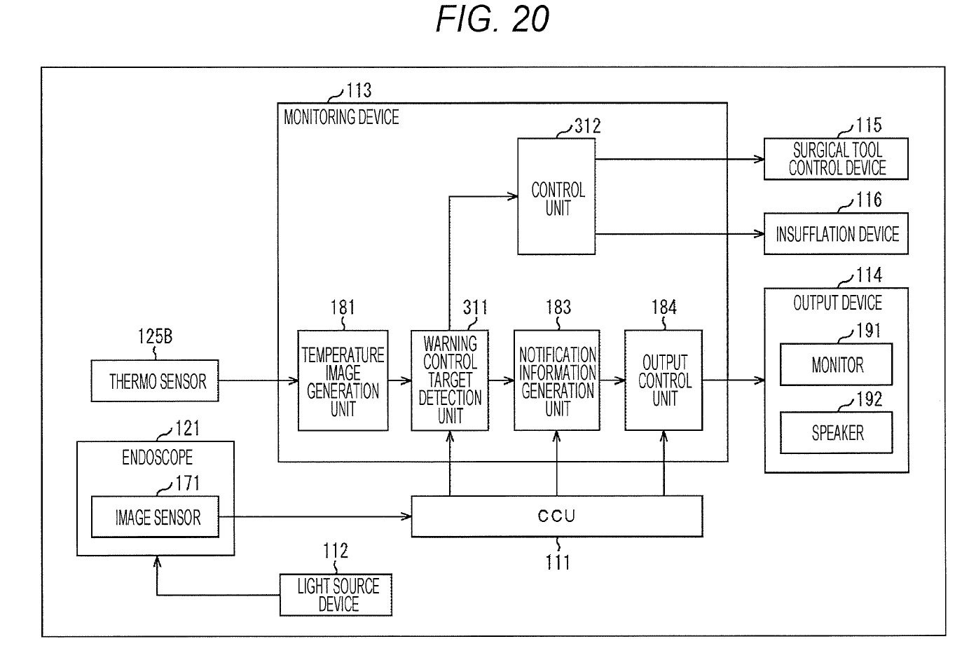

FIG. 20 is a block diagram illustrating a main configuration example of the monitoring device 113 and the like in this case. In this case also, an endoscopic surgery support system 100 basically has a configuration similar to that in a case of FIG. 3. However, in a case of FIG. 20, the monitoring device 113 is provided with a control unit 312 and further includes a warning control target detection unit 311 in place of a warning target detection unit 182 in FIG. 3.

On the basis of the temperature image supplied from the temperature image generation unit 181, the warning control target detection unit 311 detects a warning and an event subjected to the device control. In a case where the event subjected to the device control is detected, the warning control target detection unit 311 notifies a control unit 312 of that fact. Also, in a case where the event to be warned is detected, the warning control target detection unit 311 supplies that fact to a notification information generation unit 183.

When the control unit 312 is notified by the warning control target detection unit 311 that the event subjected to the device control is detected, this controls the surgical tool and the like according to the event via a surgical tool control device 115 and an insufflation device 116.

<Flow of Warning Control Process>

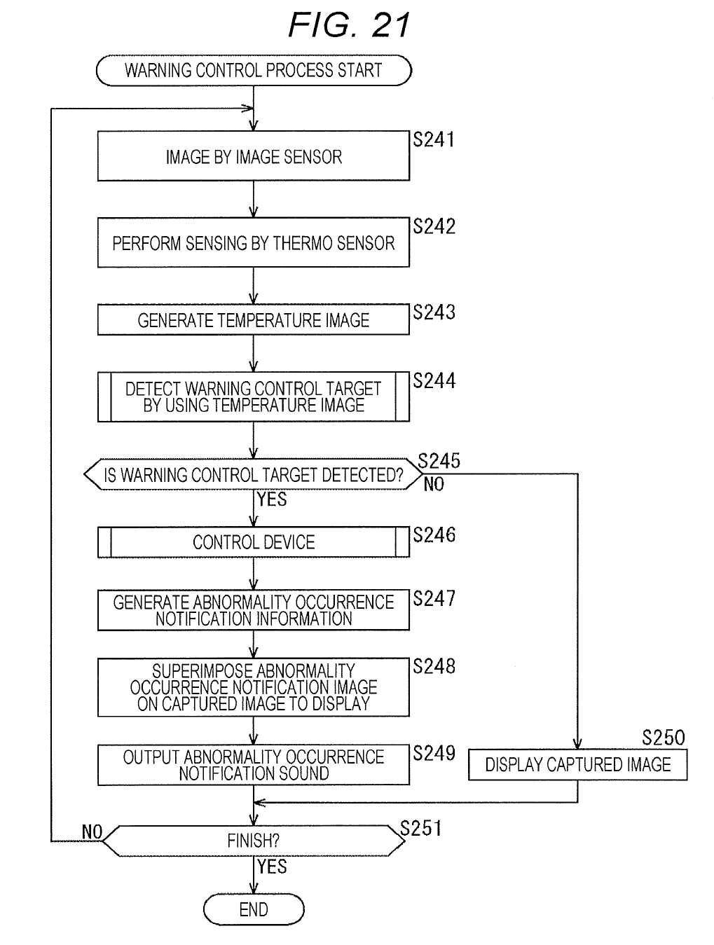

The endoscopic surgery support system 100 in this case executes a warning control process in place of a warning process. An example of a flow of the warning control process is described with reference to a flowchart in FIG. 21.

When the warning control process is started, processes at steps S241 to S243 are executed in a manner similar to that of processes at steps S101 to S103 in FIG. 4.

At step S244, the warning control target detection unit 311 detects a warning control target by using the temperature image generated at step S243. Then, at step S245, the warning control target detection unit 311 determines whether the warning control target is detected.

In a case where it is determined that the warning control target is detected, the control unit 312 controls the device in accordance with the detected event at step S246.

At step S247, the notification information generation unit 183 generates abnormality occurrence notification information for notifying that an abnormality occurs as notification information. At step S248, the output control unit 184 superimposes the abnormality occurrence notification image being the image of the abnormality occurrence notification information on the captured image obtained via the CCU 111 and allows a monitor 191 to display the same. Also, at step S249, the output control unit 184 supplies abnormality occurrence notification sound being the sound of the abnormality occurrence notification information to a speaker 192 to output.

Also, in a case where it is determined at step S245 that the warning control target is not detected, at step S250, the output control unit 184 allows the monitor 191 to display the captured image obtained via the CCU 111.

At step S251, the output control unit 184 determines whether to finish the warning control process. In a case where it is determined not to finish the warning control process, the procedure returns to step S241, and the subsequent processes are repeated. That is, while the warning process is being executed, each of the processes at steps S241 to S251 is executed. Then, in a case where it is determined at step S251 that the warning process is finished, the warning process is finished.

While the warning process is being executed, the processes at steps S241 to S251 are executed in parallel. That is, processes such as the generation of the temperature image, the detection of the warning target, the generation of the notification information, the device control, the output of the notification information and the like are performed while the image sensor 171 images and a thermo sensor 125A performs sensing.

Therefore, in the warning control target detection unit 311, the temperature image as a moving image is obtained. Therefore, the warning control target detection unit 311 detects a pattern of change in time of temperature corresponding to the event subjected to the warning control target in the moving image.

<Display Image>

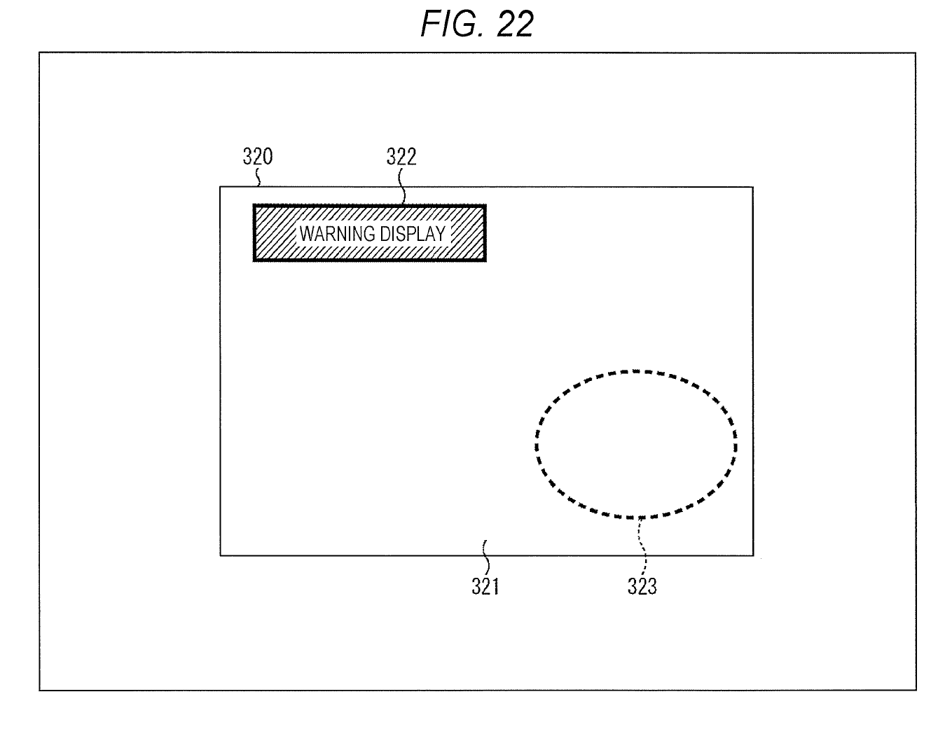

FIG. 22 illustrates an example of a display image in this case displayed on the monitor 191. A display image 320 illustrated in FIG. 22 illustrates an example of the display image including the abnormality occurrence notification image described above. As illustrated in FIG. 22, in this display image 320, the abnormality occurrence notification image is superimposed on a captured image 321.

As in the display image 320 illustrated in FIG. 22, a warning message image 222 being an image notifying of the occurrence of abnormality of the living body may be displayed as the abnormality occurrence notification image. As a result, a surgeon and the like who looks at the display image 320 may grasp the occurrence described above more easily, and also, as in the display image 320, a warning target position presentation image 323 illustrating a position where the abnormality occurs may be displayed as the abnormality occurrence notification image.

<Flow of Temperature Abnormality Detection Process>

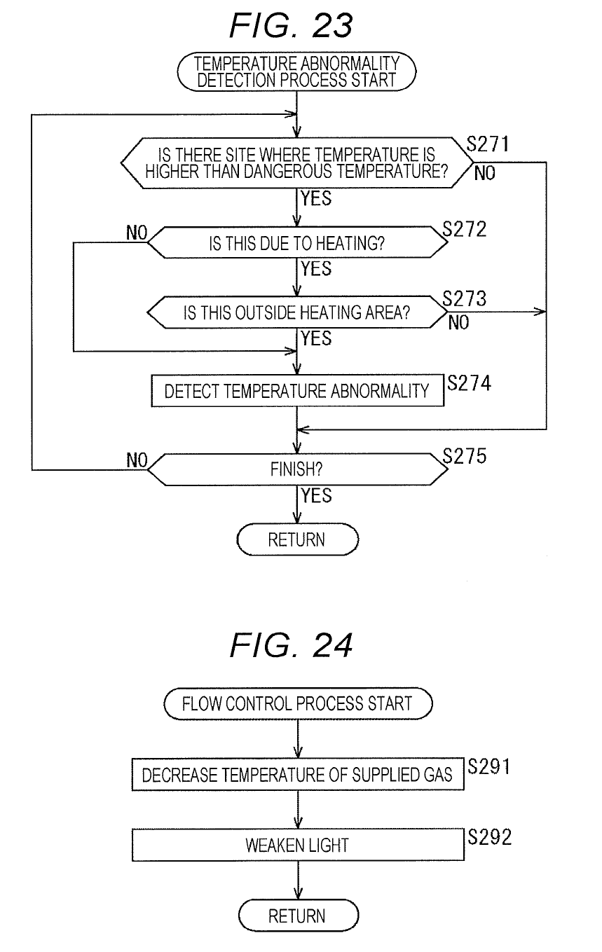

For example, the warning control target detection unit 311 may detect temperature not lower than temperature generated in the living body. For example, the temperature image may be used to detect an area at temperature that harms an organ. An example of a flow of the temperature abnormality detection process executed at step S244 in FIG. 21 is described with reference to a flowchart in FIG. 23.

When the temperature abnormality detection process is started, at step S271, the warning control target detection unit 311 determines whether there is a site where the temperature is higher than dangerous temperature. In a case where it is determined that there is the site where the temperature is higher than the dangerous temperature, the warning control target detection unit 311 determines whether the temperature increase is due to heating at step S272. In a case where it is determined that this is due to the heating, at step S273, the warning control target detection unit 311 determines whether this is out of a heating area. In a case where it is determined that this is out of the heating area, the warning control target detection unit 311 detects the temperature abnormality at step S274. Also, in a case where it is determined at step S271 that there is no site where the temperature is higher than the dangerous temperature, the procedure shifts to step S275. Also, in a case where it is determined at step S272 that this is not due to the heating, the process at step S273 is omitted, and the procedure shifts to step S274. Also, when the process at step S274 is finished, the procedure shifts to step S275.

At step S275, the warning control target detection unit 311 determines whether to finish the temperature abnormality detection process. In a case where it is determined not to finish, the procedure returns to step S271 to repeat the subsequent processes. Also, in a case where it is determined at step S275 to finish the temperature abnormality detection process, the temperature abnormality detection process is finished.

<Flow of Control Process>

Next, an example of a flow of a control process executed at step S246 in FIG. 21 is described with reference to a flowchart in FIG. 24.

When the control process is started, the control unit 312 controls the insufflation device 116 at step S291 to decrease the temperature of the supplied gas. Also, at step S292, the control unit 312 weakens the illumination and decreases the temperature in the living body on the basis of the surgical tool control device 115.

As described above, the monitoring device 113 may detect more various events on the basis of the change in time of the temperature (temperature distribution), and further control the devices and notify of the various events.

Meanwhile, in a case of a system including a plurality of light sources, the temperature control may also be performed by controlling illumination to weaken brightness and illumination to enhance the brightness. Also, the temperature control of an energy device (detection of a temperature increasing event of a peripheral part) may be performed in the similar manner.

As described above, the monitoring device 113 may notify of more various events such as warning, diagnosis, control and the like according to the event detected using the change in time of the temperature (temperature distribution).

<Other Systems>

Meanwhile, although an endoscopic surgery support system is described above as an example of a medical support system to which the present technology is applied in each embodiment, the present technology may be applied not only to the endoscopic surgery support system but also to any system. For example, this is applicable to a microscopic surgery support system supporting microscopic surgery which is abdominal surgery performed using a microscope. Although the microscope is used in place of an endoscope in the microscope surgery support system, similar to the endoscope, this microscope also includes an image sensor, irradiates a surgical site and around the surgical site with light supplied from a light source device 112, and is controlled by a CCU 111 to image the surgical site and around the surgical site.

Therefore, the present technology described above in each embodiment may be applied to such microscopic surgery support system as well as in the case of the endoscopic surgery support system, and a similar function effect may be obtained.

### 6. Sixth Embodiment

<Computer>

It is possible that the above-described series of processes is executed by hardware or executed by software. In a case where a series of processes is performed by the software, a program which forms the software is installed on a computer. Herein, the computer includes a computer built in dedicated hardware, a general-purpose personal computer, for example, capable of executing various functions by various programs installed and the like.

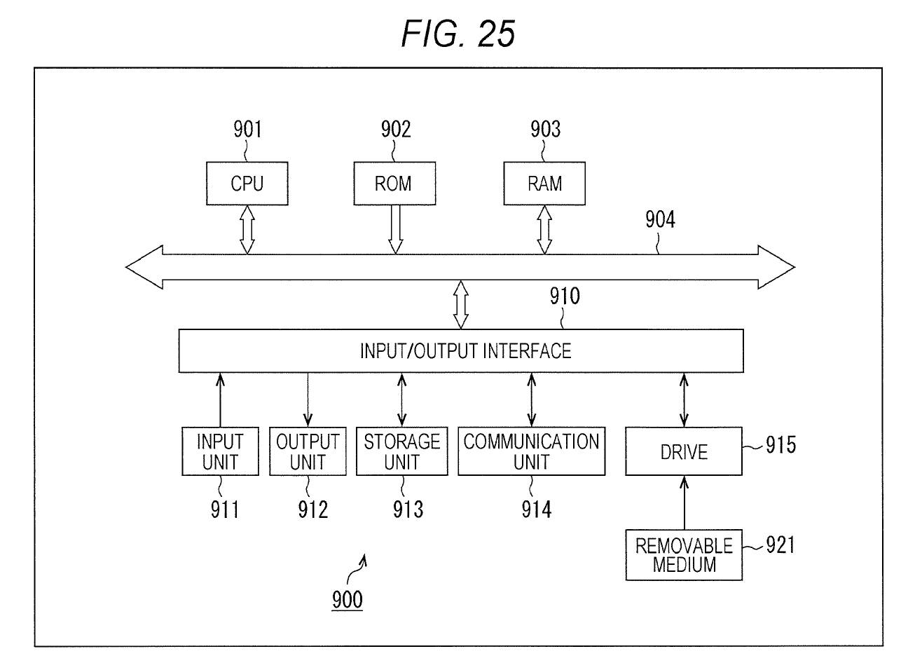

FIG. 25 is a block diagram illustrating a configuration example of the hardware of the computer which executes the above-described series of processes by the program.

In a computer 900 illustrated in FIG. 25, a central processing unit (CPU) 901, a read only memory (ROM) 902, and a random access memory (RAM) 903 are connected to one another through a bus 904.

An input/output interface 910 also is connected to the bus 904. An input unit 911, an output unit 912, a storage unit 913, a communication unit 914, and a drive 915 are connected to the input/output interface 910.

The input unit 911 includes, for example, a keyboard, a mouse, a microphone, a touch panel, an input terminal and the like. The output unit 912 includes, for example, a display, a speaker, an output terminal and the like. The storage unit 913 includes, for example, a hard disk, a RAM disk, a nonvolatile memory and the like. The communication unit 914 includes a network interface and the like, for example. The drive 915 drives a removable medium 921 such as a magnetic disk, an optical disk, a magnetooptical disk, or a semiconductor memory.

In the computer configured in the above described manner, the CPU 901 loads the program stored in the storage unit 913, for example, on the RAM 903 through the input/output interface 910 and the bus 904 to execute, so that the above-described series of processes is performed. Data required for the CPU 901 to execute the various processes also are appropriately stored in the RAM 903.

The program executed by the computer (CPU 901) may be recorded in the removable medium 921 as a package medium and the like to be applied, for example. In this case, the program may be installed on the storage unit 913 through the input/output interface 910 by mounting of the removable medium 921 on the drive 915.

The program may also be provided through a wired or wireless transmission medium such as a local area network, the Internet, digital satellite broadcasting and the like. In this case, the program may be received by the communication unit 914 to be installed on the storage unit 913.

In addition, the program may also be installed in advance on the ROM 902 and the storage unit 913.

Meanwhile, the program executed by the computer may be the program of which processes are performed in chronological order in the order described in this specification or may be the program of which processes are performed in parallel or at required timing such as when a call is issued.

Also, in this specification, a step of describing the program recorded in a recording medium includes not only the processes performed in chronological order in the described order but also the processes executed in parallel or individually which are not necessarily performed in chronological order.

Also, the process at each step described above may be executed in each device described above or any device other than the above-described device. In this case, the device which executes the process may include a function (functional block and the like) required for executing the above-described process. Also, information required for the process may be appropriately transmitted to the device.

Also, in this specification, a system is intended to mean assembly of a plurality of components (devices, modules (parts) and the like) and it does not matter whether all the components are in the same casing. Therefore, a plurality of devices stored in different casings connected through the network and one device obtained by storing a plurality of modules in one casing are the systems.

It is also possible to divide the configuration described above as one device (or processor) into a plurality of devices (or processors). Other way round, it is also possible to put the configurations described above as a plurality of devices (or processors) together as one device (or processor). Also, of course, it is possible that a configuration other than the above-described one is added to the configuration of each device (or each processor). Furthermore, it is also possible that a part of the configuration of a certain device (or processor) is included in the configuration of another device (or another processor) as long as a configuration and operation as an entire system are substantially the same.

Although preferred embodiments of the present disclosure are described above in detail with reference to the attached drawings, the technical scope of the present disclosure is not limited to such examples. It is clear that one of ordinary skill in the art of the present disclosure may conceive of various modifications or corrections within the scope of the technical idea recited in claims and it is understood that they also naturally belong to the technical scope of the present disclosure.

For example, the present technology may be configured as cloud computing in which one function is shared by a plurality of devices through a network for processing in cooperation.

Also, each step described in the above-described flowchart may be executed by one device or executed by a plurality of devices in a shared manner.

Furthermore, in a case where a plurality of processes is included in one step, a plurality of processes included in one step may be executed by one device or by a plurality of devices in a shared manner.

Also, the present technology is not limited thereto and may also be performed as any configuration mounted on a device forming such device or system, for example, a processor as a system large scale integration (LSI) and the like, a module using a plurality of processors and the like, a unit using a plurality of modules and the like, a set obtained by adding still another function to the unit (that is to say, a partial configuration of the device) and the like.

The present technology may be applied to, for example, the medical support device, an endoscope device, a microscope device, the computer controlling the devices, the medical support system including a plurality of devices, a software module thereof and the like.

Meanwhile, the present technology may also have following configurations.

(1) A medical support device including:

a detection unit which detects an event in a living body on the basis of change in time of temperature of a surgical site or around the surgical site; and

a notification information generation unit which generates notification information for notifying in accordance with the event in the living body detected by the detection unit.

(2) The medical support device according to (1),

in which the detection unit detects the event in the living body on the basis of change in time of a temperature distribution image of the surgical site or around the surgical site.

(3) The medical support device according to (2),

in which the detection unit detects a state to be warned as the event in the living body on the basis of the change in time of the temperature distribution image, and

the notification information generation unit generates a warning image to warn about the state.

(4) The medical support device according to (3),

in which the notification information generation unit generates the warning image superimposed on a captured image of the surgical site or around the surgical site to be displayed.

(5) The medical support device according to (4),

in which the detection unit detects occurrence of bleeding on the basis of the change in time of the temperature distribution image, and

the notification information generation unit generates, as the warning image, one or more of an image notifying of the occurrence of the bleeding, an image notifying of the number of times of occurrence of the bleeding, an image illustrating an occurrence site in a case where the occurrence site of the bleeding is located within a range of the captured image, and an image illustrating a direction of the occurrence site in a case where the occurrence site of the bleeding is located outside the range of the captured image.

(6) The medical support device according to (4) or (5),

in which the detection unit detects that a surgical tool after use is in a state of predetermined temperature or higher on the basis of the change in time of the temperature distribution image, and

the notification information generation unit generates, as the warning image, one or more of an image notifying of presence of the surgical tool in the state of the predetermined temperature or higher, an image notifying of the number of surgical tools, an image illustrating the surgical tool in a case where the surgical tool is located within a range of the captured image, and an image illustrating a direction of the surgical tool in a case where the surgical tool is located outside the range of the captured image.

(7) The medical support device according to any one of (4) to (6),

in which the detection unit detects a result of hemostasis on the basis of the change in time of the temperature distribution image, and

the notification information generation unit generates, as the warning image, one or more of an image notifying of the result of the hemostasis, an image notifying of the number of sites where the hemostasis is performed, an image illustrating a site where the hemostasis is performed in a case where the hemostasis is performed within a range of the captured image, and an image illustrating a direction in which the hemostasis is performed in a case where the hemostasis is performed outside the range of the captured image.

(8) The medical support device according to any one of (4) to (7),

in which the detection unit detects a damaged site of the surgical site or around the surgical site due to contact of the surgical tool not in use on the basis of the change in time of the temperature distribution image, and

the notification information generation unit generates, as the warning image, one or more of an image notifying of occurrence of the damaged site, an image notifying of the number of damaged sites, an image illustrating the damaged site in a case where the damaged site is located within a range of the captured image, and an image illustrating a direction of the damaged site in a case where the damaged site is located outside the range of the captured image.

(9) The medical support device according to any one of (4) to (8),

in which the detection unit detects a site in a state of predetermined temperature or higher of the surgical site or around the surgical site due to use of the surgical tool on the basis of the change in time of the temperature distribution image, and

the notification information generation unit generates, as the warning image, one or more of an image notifying of occurrence of the site in the state of the predetermined temperature or higher, an image notifying of the number of sites in the state of the predetermined temperature or higher, an image illustrating an occurrence position of the site in the state of the predetermined temperature or higher in a case where the site in the state of the predetermined temperature or higher is located within a range of the captured image, and an image illustrating an occurrence direction of the site in the state of the predetermined temperature or higher in a case where the site in the state of the predetermined temperature or higher is located outside the range of the captured image.

(10) The medical support device according to any one of (2) to (9),

in which the detection unit detects change in state of the living body as the event in the living body on the basis of the change in time of the temperature distribution image, and

the notification information generation unit generates a diagnosis image notifying of a diagnosis result based on the change in state.

(11) The medical support device according to (10), further including:

a heating unit which heats the living body,

in which the detection unit detects the change in state of the living body after being heated by the heating unit.

(12) The medical support device according to (11),

in which the detection unit performs detection of a subsegment of a liver, detection of a lesion, detection of a blood vessel, detection of a lymphatic vessel, identification of bone and nerve, or detection of a cancer cell, and

the notification information generation unit generates an image illustrating the subsegment, the lesion, the blood vessel, the lymphatic vessel, the bone, the nerve, or the cancer cell detected by the detection unit so as to be identifiable superimposed on a captured image of the surgical site to be displayed as the diagnosis image.

(13) The medical support device according to any one of (1) to (12),

in which the detection unit further detects the event in the living body on the basis of a sensing result other than the change in time of the temperature of the surgical site or around the surgical site.

(14) The medical support device according to (13),

in which the detection unit detects the event in the living body on the basis of the change in time of the temperature of the surgical site or around the surgical site and a stereo image of the surgical site or around the surgical site.

(15) The medical support device according to any one of (1) to (14), further including:

a temperature sensor which detects temperature of the surgical site or around the surgical site,

in which the detection unit detects the event in the living body on the basis of the change in time of the temperature of the surgical site or around the surgical site detected by the temperature sensor.

(16) The medical support device according to any one of (1) to (15), further including:

a display unit which displays an image of the notification information generated by the notification information generation unit.

(17) The medical support device according to any one of (1) to (16), further including:

a control unit which controls the surgical tool according to the event in the living body detected by the detection unit.

(18) The medical support device according to any one of (1) to (17),

in which the control unit controls temperature of gas supplied into the living body or brightness of illumination light emitted into the living body.

(19) A medical support method of a medical support device including:

detecting an event in a living body on the basis of change in time of temperature of a surgical site or around the surgical site, and

generating notification information notifying according to the detected event in the living body.

(20) A medical support system including: a temperature sensor; a medical support device; and a display device,

the temperature sensor configured to measure temperature of a surgical site or around the surgical site,

the medical support device including:

a detection unit which detects an event in a living body on the basis of change in time of temperature of the surgical site or around the surgical site measured by the temperature sensor; and

a notification information generation unit which generates notification information notifying according to the event in the living body detected by the detection unit, and

the display device configured to display an image of the notification information generated by the notification information generation unit.

### REFERENCE SIGNS LIST

100 Endoscopic surgery support system
111 CCU
112 Light source device
113 Monitoring device
114 Output device
115 Surgical tool control device
116 Insufflation device
121 Endoscope
122 Energy device
123 Forceps
124 Insufflation needle
125 Monitoring sensor
125A Thermo sensor
125B Stereo image sensor
131 Trocar
141 Patient bed
151 Patient
161 Affected site
162 Imaging area
163 Sensing area
171 Image sensor
181 Temperature image generation unit
182 Warning target detection unit
183 Notification information generation unit
184 Output control unit
191 Monitor
192 Speaker
211 Organ
212 Area
220 Display image
221 Captured image
222 Bleeding warning message image
223 Bleeding position presentation image
224 Bleeding direction presentation image
230 Display image
231 Captured image
232 Residual heat warning message image
233 Residual heat warning image
240 Display image
241 Captured image
242 Biological temperature abnormality warning message image
243 Biological temperature abnormality warning image
251 Warning target detection unit
261 Heater
271 Heating control unit
272 Diagnosis unit
290 Display image
291 Captured image
292 Subsegment identification image
311 Warning control target detection unit
312 Control unit
320 Display image
321 Captured image
322 Warning message image
323 Warning target position presentation image
900 Computer

1. A medical support device comprising:
a detection unit which detects an event in a living body on the basis of change in time of temperature of a surgical site or around the surgical site; and
a notification information generation unit which generates notification information for notifying in accordance with the event in the living body detected by the detection unit.

2. The medical support device according to claim 1,
wherein the detection unit detects the event in the living body on the basis of change in time of a temperature distribution image of the surgical site or around the surgical site.

3. The medical support device according to claim 2,
wherein the detection unit detects a state to be warned as the event in the living body on the basis of the change in time of the temperature distribution image, and
the notification information generation unit generates a warning image to warn about the state.

4. The medical support device according to claim 3,
wherein the notification information generation unit generates the warning image superimposed on a captured image of the surgical site or around the surgical site to be displayed.

5. The medical support device according to claim 4,
wherein the detection unit detects occurrence of bleeding on the basis of the change in time of the temperature distribution image, and
the notification information generation unit generates, as the warning image, one or more of an image notifying of the occurrence of the bleeding, an image notifying of the number of times of occurrence of the bleeding, an image illustrating an occurrence site in a case where the occurrence site of the bleeding is located within a range of the captured image, and an image illustrating a direction of the occurrence site in a case where the occurrence site of the bleeding is located outside the range of the captured image.

6. The medical support device according to claim 4,
wherein the detection unit detects that a surgical tool after use is in a state of predetermined temperature or higher on the basis of the change in time of the temperature distribution image, and
the notification information generation unit generates, as the warning image, one or more of an image notifying of presence of the surgical tool in the state of the predetermined temperature or higher, an image notifying of the number of surgical tools, an image illustrating the surgical tool in a case where the surgical tool is located within a range of the captured image, and an image illustrating a direction of the surgical tool in a case where the surgical tool is located outside the range of the captured image.

7. The medical support device according to claim 4,
wherein the detection unit detects a result of hemostasis on the basis of the change in time of the temperature distribution image, and
the notification information generation unit generates, as the warning image, one or more of an image notifying of the result of the hemostasis, an image notifying of the number of sites where the hemostasis is performed, an image illustrating a site where the hemostasis is performed in a case where the hemostasis is performed within a range of the captured image, and an image illustrating a direction in which the hemostasis is performed in a case where the hemostasis is performed outside the range of the captured image.

8. The medical support device according to claim 4,
wherein the detection unit detects a damaged site of the surgical site or around the surgical site due to contact of the surgical tool not in use on the basis of the change in time of the temperature distribution image, and
the notification information generation unit generates, as the warning image, one or more of an image notifying of occurrence of the damaged site, an image notifying of the number of damaged sites, an image illustrating the damaged site in a case where the damaged site is located within a range of the captured image, and an image illustrating a direction of the damaged site in a case where the damaged site is located outside the range of the captured image.

9. The medical support device according to claim 4,
wherein the detection unit detects a site in a state of predetermined temperature or higher of the surgical site or around the surgical site due to use of the surgical tool on the basis of the change in time of the temperature distribution image, and
the notification information generation unit generates, as the warning image, one or more of an image notifying of occurrence of the site in the state of the predetermined temperature or higher, an image notifying of the number of sites in the state of the predetermined temperature or higher, an image illustrating an occurrence position of the site in the state of the predetermined temperature or higher in a case where the site in the state of the predetermined temperature or higher is located within a range of the captured image, and an image illustrating an occurrence direction of the site in the state of the predetermined temperature or higher in a case where the site in the state of the predetermined temperature or higher is located outside the range of the captured image.

10. The medical support device according to claim 2,
wherein the detection unit detects change in state of the living body as the event in the living body on the basis of the change in time of the temperature distribution image, and
the notification information generation unit generates a diagnosis image notifying of a diagnosis result based on the change in state.

11. The medical support device according to claim 10, further comprising:
a heating unit which heats the living body,
wherein the detection unit detects the change in state of the living body after being heated by the heating unit.

12. The medical support device according to claim 11,
wherein the detection unit performs detection of a subsegment of a liver, detection of a lesion, detection of a blood vessel, detection of a lymphatic vessel, identification of bone and nerve, or detection of a cancer cell, and
the notification information generation unit generates an image illustrating the subsegment, the lesion, the blood vessel, the lymphatic vessel, the bone, the nerve, or the cancer cell detected by the detection unit so as to be identifiable superimposed on a captured image of the surgical site to be displayed as the diagnosis image.

13. The medical support device according to claim 1,
wherein the detection unit further detects the event in the living body on the basis of a sensing result other than the change in time of the temperature of the surgical site or around the surgical site.

14. The medical support device according to claim 13,
wherein the detection unit detects the event in the living body on the basis of the change in time of the temperature of the surgical site or around the surgical site and a stereo image of the surgical site or around the surgical site.

15. The medical support device according to claim 1, further comprising:
a temperature sensor which detects temperature of the surgical site or around the surgical site,
wherein the detection unit detects the event in the living body on the basis of the change in time of the temperature of the surgical site or around the surgical site detected by the temperature sensor.

16. The medical support device according to claim 1, further comprising:
a display unit which displays an image of the notification information generated by the notification information generation unit.

17. The medical support device according to claim 1, further comprising:
a control unit which controls the surgical tool according to the event in the living body detected by the detection unit.

18. The medical support device according to claim 1,
wherein the control unit controls temperature of gas supplied into the living body or brightness of illumination light emitted into the living body.

19. A medical support method of a medical support device comprising:
detecting an event in a living body on the basis of change in time of temperature of a surgical site or around the surgical site, and
generating notification information notifying according to the detected event in the living body.

20. A medical support system comprising: a temperature sensor; a medical support device; and a display device,
the temperature sensor configured to measure temperature of a surgical site or around the surgical site,
the medical support device including:
a detection unit which detects an event in a living body on the basis of change in time of temperature of the surgical site or around the surgical site measured by the temperature sensor; and
a notification information generation unit which generates notification information notifying according to the event in the living body detected by the detection unit, and
the display device configured to display an image of the notification information generated by the notification information generation unit.
